(19) United States
(12) Patent Application Publication
(10) Pub. No.: US 2026/0018254 A1
(43) Pub. Date: Jan. 15, 2026

(54) METHOD FOR DETERMINING A TARGET SYNTHESIS SPECIFICATION

(71) Applicant: BASF SE, Ludwigshafen am Rhein (DE)

(72) Inventors: Astrid Niederle, Freeport, TX (US); Philipp Eiden, Ludwigshafen (DE); Andree Lischewski, Uster (CH); Volker Settels, Ludwigshafen (DE); Ning Wang, Ludwigshafen (DE); Frederik Sandfort, Ludwigshafen (DE)

(21) Appl. No.: 18/862,616

(22) PCT Filed: May 5, 2023
(86) PCT No.: PCT/EP2023/062010

§ 371 Date: Nov. 4, 2024

(30) Foreign Application Priority Data
May 5, 2022 (EP) 22171863.8

(51) Int. Cl. G16C 20/10; G16C 20/70

(52) U.S. Cl. CPC G16C 20/10; G16C 20/70

(57) ABSTRACT

The invention refers to a method for determining a target molecule. A target property and a digital representation of a potential target molecule is provided. Then a model is utilized for determining a property of a potential target molecule. The model has been parameterized based on an explored data set and an unexplored data set. The explored data set comprises a property for a plurality of explored molecules and molecule characterizing parameter values for the plurality of explored molecules. The unexplored data set comprising characterizing parameter values for a plurality of unexplored molecules. The determined property of the potential target molecule is compared with the target property. Based on the comparison, either i) the potential target molecule is determined as the target molecule, or ii) a new potential molecule is determined and the determination of the property repeated. The determined target molecule is then provided.

### FIELD OF THE INVENTION

The invention relates to a method, an apparatus and a computer program product for determining a synthesis specification and/or a molecular structure of a target molecule comprising a target technical application property. Further, the invention refers to a method, an apparatus and a computer program product for generating a machine learning based determination model that can be utilized for determining a technical application property of a molecule. Moreover, the invention refers to a method, apparatus and computer program product for determining molecule characterizing parameters for training a machine learning based determination model that can be utilized to determine a technical application property of a molecule. Furthermore, the invention refers to interface methods, interface apparatuses, and interface computer program products providing an interface for the above methods, apparatuses and computer program products.

### BACKGROUND OF THE INVENTION

Generally, different molecules, in particular, polymers, produced by the chemical industry are widely used in industrial and/or daily use products with a broad range of application properties. For developing new molecules or for utilizing known molecules in new application contexts, it is often useful to have knowledge of at least some of the technical application properties, for instance, a heat insulating factor, a hardness, or a reflectivity, of the molecule in advance. For such purposes, in many cases determination models are utilized that are, for instance, based on machine learning principles and are trained to determine for a given molecule a respective technical application property.

### SUMMARY OF THE INVENTION

Developing determination models such that they allow for a high accuracy in a specific application context is often a difficult, error-prone and time-consuming process. In particular, the providing of a training data set that allows for training a determination model to be applicable in a predetermined application context is often only based on expert knowledge and trial-and-error processes in which a potential training data set is used for training a model and based on the result of the training, i.e. based on the possibilities of the trained determination model, the training data set is then revised again and again. Since providing such a training data set is always associated with measuring a plurality of characteristics and application properties for the molecules being part of the training data set, this process also leads to a high consumption of laboratory resources, for instance, material resources, human resources, computational resources, etc. Moreover, a generally known training processes of determination models are very time and computational resource consuming such that avoiding unproductive training, i.e. training leading to an unsuitable model, would be very helpful. Thus, it would be advantageous if it would be possible to train determination models, in particular, to provide training data for training determination models, more effectively, i.e. with less resource consumption, and more objectively, i.e. less based on expert knowledge. Further, typical training processes are based on historical molecule data for a specific application. However, for new applications the respectively trained models are often less suitable and a completely new model has to be trained with the respective above described time and computational resource intensive training process. Thus, it would also be advantageous if also already during the training process a respective application possibility to new applications could be taken into account making the models more generally applicable.

It is an object of the present invention to provide methods, apparatuses and computer program products for a) determining target molecules comprising one or more target application properties, b) for training determination models utilized for determining the target molecules, and c) for providing training data sets for training determination models, wherein the utilized determination models can be provided, in particular, trained more effectively and more objectively.

In a first aspect of the present invention, a computer implemented method for determining a synthesis specification and/or molecular structure of a target molecule, in particular, target polymer, comprising a target technical application property is presented, wherein the method comprises i) providing a target technical application property, ii) providing a digital representation of a potential target molecule indicative of or associated with characterizing parameters, iii) utilizing a trained determination model for determining a technical application property of the potential target molecule, wherein the trained determination model has been parameterized based on an explored data set and an unexplored data set such that the trained determination model is adapted to determine a technical application property of a molecule based on one or more molecule characterizing parameters of the molecule, wherein the explored data set comprises a) a technical application property for a plurality of explored molecules and b) a plurality of molecule characterizing parameter values for a plurality of molecule characterizing parameters associated with synthesis specifications and/or molecular structures corresponding to each of the plurality of explored molecules, and the unexplored data set comprising a plurality of molecule characterizing parameter values for a plurality of molecule characterizing parameters associated with synthesis specifications and/or molecular structures corresponding to a plurality of unexplored molecules, iv) comparing the determined technical application property of the potential target molecule with the target technical application property and, based on the comparison, either i) determining the potential target molecule as the target molecule, or ii) providing a new potential target molecule and repeating the determination of the technical application property utilizing the new potential target molecule, v) providing the determined target molecule. Since the trained determination model is parameterized not only based on an explored data set, i.e. a data set comprising technical application properties, but also based on an unexplored data set, it has been found by the inventors that the determination model can be trained more effectively and more objectively. In particular, an unexplored data set can be easily generated with few computational resources and utilized for defining possible technical applications of the determination model, for instance, the characterizing parameters that can be used for determining the technical application property and/or the molecules to which the trained determination model can be applied. A such generated unexplored data set can then be utilized for tailoring the training data set based on the explored data set to the such determined technical application context. This allows to decrease the amount of trial-and-error runs for training the determination model and also the amount of additional measurements to determine further explored data. Moreover, an applicability domain of the trained model can be increased based on the unexplored data set and thus the such trained model is more generally applicable. Moreover, the influence of expert knowledge on the tailoring of the training data set can be strongly decreased leading to a more objective training.

The method refers to a computer implemented method and thus can be performed by a general or dedicated computer, or network of computers, adapted to perform the method, for instance, by executing a respective computer program. The method is configured for determining a target molecule comprising a target technical application property. Generally, the term “molecule” relates to a group of two or more atoms held together by chemical bonds, wherein all bonds between non-hydrogen-atoms are defined. The bonds could have stochastic character like in random co-polymers, for example. Moreover, each molecule as used herein is associated with a specific molecular structure and/or synthesis specification such that the molecular structure and/or synthesis specification defines the molecule. However, a molecule could be associated with different molecular structures. For example, this would be the case for a polymer with a molar mass distribution. Accordingly, providing a molecule generally also comprises providing the respective molecular structure and/or synthesis specification of the molecule. In an embodiment, the molecule is a small molecule, wherein herein a small molecule is defined as a molecule that is present in the environment in a form that allows to completely describe the molecule using simple structural formulas, that contain the relevant information. A simple molecular formula refers to molecules that can be described by covalent bonds between the atoms of the molecule. However, molecules could consist out of isomers and could be partly protonated. Examples, where this is not the case, are e.g. systems with dynamic equilibria between several forms like monomer and oligomers as in the case of several inorganic acids, or ionic species with very localized charge that strongly interacts with a solvent, e.g. via hydrogen bonding. Preferably, a small molecule is defined by a molecular weight of less than 600 g/mol, more preferably of less than 300 g/mol. Preferably, the target molecule is a target polymer, and the method is configured for determining a target synthesis specification of the target polymer. Generally, a synthesis specification is defined as an instruction on how a molecule, in particular a polymer, can be synthesized. In particular, for a polymer the synthesis specification indicates the starting substances and the respective parameters for polymerization from the starting substances.

A technical application property can generally refer to any property of a molecule and/or a substance consisting at least partly of the molecule, for example, a formulation or mixture comprising the molecule, that allows to assess a technical applicability of the respective molecule as provided after its synthesis. Preferably, the technical application property is a property that is associated with a substance comprising or consisting of the molecule. In particular, the technical application property can be defined by an intended technical application of the molecule and/or a substance consisting at least partly of the molecule. In particular, a technical application property can be distinguished from a non-technical application property in that a technical application property directly allows for a determination whether a molecule is suitable in a technical application, whereas a non-technical application property, for instance, a bonding strength between two atoms of the molecule, does not directly allow for determining a technical application property. Preferably, the technical application property comprises at least one of mechanical properties, optical properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. An optical property can generally comprise any of coloration, turbidity, opaqueness, lucidity, reflection, appearance, absorption, scattering, color strength, colour hue, colour saturation, colour intensity, cloud point, matting degree, optical density, spectra, refractive index. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, and charge density. The chemical property can comprise any of chemical resistance, reaction timing, demolding time, growing, hard/soft segment content, crystallinity, reaction temperature, reaction pressure, decomposition, thermal decomposition, photodegradation, acidity, pKa, pH, mois-ture/water content, flammability, burning rate, selfignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, in particular, resistance against a pathogenic virus, bacterium, fungus, plant or animal or developmental stage of said pathogen, tolerance against environmental parameters, e.g. drought tolerance, resistance against enzy-matic degradation, e.g. protease resistance, lipase resistance, amylase resistance, hydro-lase resistance, pesticide resistance, toxicity, biotransformation, ecotoxicology, sensitization, in particular, allergenicity, bacterial count, enzyme activity, substrate specificity, co-factor dependence, product specificity, substrate and/or product inhibition, dissociation constant, Michaelis-Menten-kinetics values, activity/stability at or in different: pH, temperature, pressure, organic solvent concentration, carrier formulations, encapsulation formulations; distribution in environment, compartmentalization, bioaccumulation, biological expo-sure LD50, mutagenicity.

In a first step, the method comprises providing a target technical application property. Generally, in this invention if not stated otherwise the providing of a quantity also refers to the providing of a quantification, e.g. a value or value range, of the quantity. Thus, the providing of the target technical application property comprises also providing a quantification of the target technical application property, for example a value or value range of the target technical application property. In particular, the providing can refer to receiving the target application property from an input of a user applying, for instance, a respective input unit. Moreover, the providing can also refer to accessing a storage unit on which a target application property is already stored. Further, the providing can also comprise receiving a target application property, for instance, via a network connection from other sources and providing the received target application property. Generally, the target application property can refer to one target value, for instance, a specific hardness of a molecule, or can refer to a value range that should be met by the molecule. Moreover, the target application property can also refer to any kind of target function, for instance, a timely sequence of a property under a changing environmental condition, like a hardness under changing temperature conditions. Such more complex target application property can be advantageous in cases in which the application of the molecule comprises different environmental conditions, for example, different temperatures. The target molecule then refer to a molecule that provides the respective target technical application property when provided in a respective form, for example, as pure substance, or mixture. In particular, the target molecule provides the respective target technical application property when synthesized according to the corresponding synthesis specification and/or molecular structure.

Further, the method comprises providing a digital representation of a potential target molecule indicative of or associated with characterizing parameters of the potential target molecule. Generally, a digital representation is defined as data provided in a form and structure that allows a processing in a digital system, in particular, a computing system, for example, a digital representation can be a text string, and ID, an image, a chemical formula, etc. Preferably, the digital representation comprises the characterizing parameters of the potential target molecule. Generally, the characterizing parameters of a molecule quantify one or more characteristics intrinsic to the molecule itself, in particular, physicochemical characteristics of the molecule itself. Thus, the characterizing parameters refer to the molecule itself whereas the technical application property refers to a property of the molecule in its technical application form, for instance, as substance comprising or consisting of the molecule. Preferably, the characterizing parameters are associated with a synthesis specification and/or molecular structure. Here “associated with” means that the characterizing parameters can directly refer to the synthesis specification and/or molecular structure, for example, as structural or process parameter, or can be derived from the synthesis specification and/or molecular structure, for example, as computed physicochemical properties that can be calculated from the synthesis specification and/or molecular structure. Preferably, for molecules that can be defined by a simple structural formula the characterizing parameters are associated with a molecular structure, since the molecular structure defined the molecule unambiguously. However, also in this case a synthesis specification allows to derive suitable characterising parameters. In case of more complex molecules that can often exist in a plurality of different structures or for which it is often difficult to determine the exact molecular structure, in particular, for polymers, it is preferred that the characterizing parameters are associated with the synthesis specification. However, also in these cases often at least some parts of the molecular structure can be defined and then utilized for determining characterizing parameters. Generally, the characterizing parameters can comprise any of process parameters, recipe parameters, synthesis routes or physicochemical parameters of the molecule. Process parameters, in this context, quantify the production process of the respective molecule and can be derived from a synthesis specification of the molecule. Since the specific process parameters lead to the production of a specific molecule, the process parameters also characterize the molecule itself. For example, the process parameters can refer to any of a temperature profile, a pressure, a stirring power, synthesis steps, etc. The recipe parameters generally quantify the production of the molecule itself, in particular, the substances utilized for producing the molecule. Such substances can refer to starting substances, like in case of the molecule being a polymer respective prepolymers, from which the molecule is produced. However, the substances can also refer to auxiliary substances, like catalysts. In this context, the recipe parameters quantify the influence of these substances on the produced molecule and thus also characterize the molecule itself. For example, the recipe parameters can refer to any of an amount of specific chemical substance, an amount of specific additives, a mixing ration between different substance, or a used solvent. Preferably, the digital representation comprises molecule physicochemical parameters, preferably, referring to molecule descriptors, i.e. computed physicochemical molecule properties, wherein the molecule physicochemical parameters are indicative of physicochemical characteristics of the molecule. In particular, the molecule physicochemical parameters are indicative of parameters quantifying the physicochemical characteristics of the molecule. In this context, the term “physicochemical characteristics” refers to physical and/or chemical characteristics of the molecule. For example, the physicochemical parameters can refer to any of a molar mass distribution, a viscosity, a degree of protonation, etc. However, the digital representation can also be provided such that it allows to derive the molecule physicochemical parameters, for instance, by providing a representation of the molecule for which respective molecule physicochemical parameters are already stored or can be determined, for instance, by respective molecule descriptor calculations. Preferably, the digital representation comprises at least one of a recipe, a structural formula, a brand name, an IUPAC name, a chemical identifier and a CAS number of a molecule.

In a preferred embodiment, the target molecule is a polymer and the characterizing parameters are indicative of parameters quantifying the physicochemical characteristics of subgroups of the polymer. In this embodiment, the digital representation can also be provided such that it allows to derive the characterizing parameters by determining subgroups of the polymer and to determine the characterizing parameters based on physicochemical characteristics of the determined subgroups. Generally, a subgroup refers to a part of the polymer, wherein all subgroups of a polymer together form the polymer. For example, a subgroup can refer to a part of the polymer, wherein the subgroups are linked together suc-cessively along a chain or network to form the polymer. Preferably, the subgroups of the polymer refer to repeating units that describe a part of the polymer which when repeated produces the complete polymer chain. However, in some cases, a subgroup can also refer to a single part of the polymer that is not repeated. Moreover, it is preferred that the subgroups comprise parts that are repeated, for example, a subgroup of a polymer can comprise a repeating core also present in other subgroups and further additional parts that are not present in other subgroups. Preferably, the subgroups refer to at least one of polymerized monomers or oligomer fragments. More preferably, the subgroups refer to polymerized monomers. In this context, polymerized monomers refer to monomers after their polymerization sometimes also called “mer unit” or “mer”. In particular, polymerized monomers do not refer to monomers, i.e. raw materials, as present in a reaction mixture before polymerization, but refer to repeating units derived from monomers that have been changed during or after the polymerization. Thus, subgroup descriptors determined for polymerized monomers are different from subgroup descriptors determined for unreacted monomers before polymerization. It has been found by the inventors that in particular the polymerized monomers allow to determine polymer descriptors from the subgroup descriptors of the polymerized monomers that allow the determination model to accurately determine the technical application properties for the polymer. In a preferred embodiment, the digital representation of the polymer comprises subgroups provided as molecular model which is indicative of a chemical structure of the subgroup after its polymerization. Even more preferably, the molecular model of a subgroup is determined in a way that is suited for quantum chemical computations regarding a number of atoms and their connectivity that is representative of the properties of the subgroup within the polymer. Moreover, additionally or alternatively to a molecular model of a subgroup treating the subgroup as a monomer structure, also a molecular model referring to an oligomer model can be utilized that takes into account ef-fects of neighbouring molecular structures of the subgroup in the polymer.

In an embodiment, if the digital representation of the molecule does not directly comprise the characterizing parameters, it is preferred that the characterizing parameters are determined form an indicated molecular structure and/or synthesis specification of the provided molecule. If the molecular structure and/or a synthesis specification is not provided as part of the digital representation, the molecular structure and/or synthesis specification can be derived from the information on the molecule itself, for instance, by utilizing respective molecule databases or known methods for synthesis specification determination, like data-driven retro-synthesis path planner. Moreover, the molecular structure and/or synthesis specification can also be requested to be provided in this case by a user. If the molecule is a polymer it is preferred that the characterizing parameters are determined based on a provided synthesis specification instead of a molecular structure that can often not be determined for a polymer. Further, in this case if the characterizing parameters are based on characterizing parameters of subgroups, it is preferred that the characterizing parameters are determined by first determining the subgroups of the polymer. For example, respective subgroups of the polymer can be determined utilizing known methods. In particular, it is preferred that the subgroups are determined such that between atoms of different subgroups in the polymer the bond is as least polarized as possible and, preferably, with a bond order as small as possible (e.g. a CC single bond). Additionally, it is preferred that the subgroups representing a polymer comprise the same number of active non-hydrogen-atoms then the polymer. Besides the active atoms, a subgroup can also contain further atoms, which can be ignored during computing the physicochemical parameters of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languishes like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of monomers of the polymer are directly derivable and, for example, RDkit can be used to determine from the SMILES of the monomers the SMILES, i.e. the number and connectivity of the atoms, of the subgroups.

The determined subgroups of the polymer are associated with subgroup characterizing parameters indicative of parameters quantifying, in particular, physicochemical characteristics of the subgroups in the polymer. In particular, it is preferred that if the characterizing parameters are not directly provided by the digital representation, the characterizing parameters are determined by determining respective subgroup characterizing parameters for each of the subgroups and to determine the characterizing parameters based on the subgroup characterizing parameters of the subgroups, for instance, by averaging. Thus, the in this embodiment method preferably comprises first providing or determining for the polymer the subgroups from the digital representation, then to determine or provide the subgroup characterizing parameters, i.e. values of the parameters quantifying the characterizing parameters, of the subgroups, and then to determine the polymer characterizing parameters based on the subgroup characterizing parameters of each polymer.

Preferably, the molecule characterizing parameters comprise physicochemical parameters referring to at least one of constitutional descriptors, count descriptors, list of structural fragments, fingerprints, graph invariants, 3D-descriptors and/or higher dimensional descriptors that are indicative of parameters quantifying physicochemical characteristics of the molecule. Moreover, in the case of polymers it is preferred that the molecule characterizing parameter comprise as physicochemical parameter a molar mass distribution of the polymer. In the case of polymers, it is further preferred that the molecule characterizing parameter comprise as process parameter a temperature profile. Furthermore, it is preferred that the molecule characterizing parameter comprise as recipe parameter an amount of respective ingredients. Generally, the molecule characterizing parameters can also preferably comprise any combination of the above or below described possible parameters.

In a preferred embodiment the molecule characterizing parameters comprise 3D descriptors, in particular computed physicochemical properties, and or fingerprints. If the molecule is a polymer, the molecule characterizing parameters can be derived from the subgroup parameters, thus, also the subgroup characterizing parameters can refer to the same characterizing parameters as stated above. Generally, the characterizing parameters can also be derived without utilizing subgroups, for instance, by simulations of the whole molecule. In the following possible characterizing parameters are defined in more detail. Also in these cases the defined characterizing parameters can refer directly to the molecule characterizing parameters or, optionally for the polymer embodiment, to the subgroup characterizing parameters.

A constitutional descriptor can refer to any of a potential, average molecular weight, poly-dispersity, charge, spin, boiling point, melting point, enthalpy of fusion, dissociation constant, Hansen parameter, protic, polar and dispersive contributions, Abraham parameter, retention index, TPSA, torsion angle, branching degree, nucleotide and/or amino acid composition, nucleotide and/or amino acid sequence, nucleotide and/or amino acid sequence conservation, isoelectric point, glycosylation pattern, receptor binding constant, Inhibitor constant. A count descriptor can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers. Molecule descriptors referring to a list of structural frag-ment descriptors can refer to at least one of a list of molecular fractions, a list of functional groups, a list of bonds, and a list of atoms. Fingerprint descriptors comprise preferably, at least one of MACCS keys, preferably, in bit format or total amount format, Morgan and other circular fingerprints, preferably, in bit format or total amount format, topological torsion, atom pairs, infrared and related spectra, fingerprint count, PubChem fingerprint, sub-structure fingerprint, and Klekota-Roth fingerprint. Graph invariants/topological indices descriptors comprise preferably at least one of topostructural indices and topochemical indices. In a preferred embodiment the characterizing parameters comprise 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the molecule characterizing parameters refer to 3D descriptors comprisesing at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or nonpolar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of molecule, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution.

In a next step, a trained determination model is utilized for determining a technical application property of the molecule. Preferably, the utilizing of the determination model can comprise providing or receiving the determination model, for instance, from a respective storage unit and utilizing the provided or received determination model. The trained determination model has been parameterized based on an explored data set and an unexplored data set such that the trained determination model is adapted to determine a technical application property of a molecule based on one or more of the molecule characterizing parameters of the molecule. Generally, the parameterization of the trained determination model refers to the determination of the parameters of the determination model in a training process, thus the determination model is trained based on an explored data set and an unexplored data set. The term “such that” is to be interpreted here that the parameterization, i.e. training, adapts and thus enables the determination model to determine a technical application property of a molecule based on one or more of the molecule characterizing parameters of the molecule. In particular, the determination model is a data driven model, wherein the term “data driven” is used to emphasize that the model is mainly based on respective data input and not, for instance, on intuition, personal experience or knowledge. Preferably, the determination model is based on known machine learning algorithms, like neural networks, regression models, classification algorithms, etc. It has been found that for most applications in this context, in particular, regression models based on Linear Regression, Random Forests, Lasso, Boosted Trees, Ridge Regression and MARS algorithms are suitable, whereas for classification models, in particular, Random Forests, Lo-gistic Regression, and SVM algorithms are suitable. Generally, the determination model is parameterized during a training process, wherein in the training process an explored data set and an unexplored data set are utilized for the training of the determination model.

Generally, the explored data set comprises a) a technical application property for a plurality of explored molecules and b) a plurality of molecule characterizing parameter values for a plurality of molecule characterizing parameters associated with synthesis specifications and/or molecular structures corresponding to each of the plurality of explored molecules. Thus, an explored data set refers to a data set comprising only molecules for which at least once at least one technical application property has been determined. Generally, an explored molecule refers to a molecule for which at least one technical application property has been determined. The term “associated with” in the formulation “molecule characterizing parameters associated with synthesis specifications and/or molecular structures” is to be interpreted here as “define by” or “derivable from” the respective synthesis specification and/or molecular structure as already described above with respect to the characterizing parameters of the potential target molecule. The technical application property of an explored molecule can be determined in any known manner, for example, based on experi-mental data and measurements, based on simulations or theoretical considerations. Preferably, the technical application property refers to a measured technical application property. For example, for measuring the technical application property a respective molecule can be synthesized and the respective technical application property measured in a suitable measurement process. However, the technical application property can also be determined based on respective physical or chemical calculations or based on respective simulations of the molecule. The molecule characterizing parameter values associated with the molecule can also be measured but can also be determined in any other known way, for instance, can be calculated, as already described above. In an embodiment, the explored data set can further comprise producability information for one or more of the plurality of molecules of the explored data set, wherein the producability information is indicative of which technical application property of the molecule can be measured. For example, during a synthesis of a molecule it can be determined that the molecule is generally not producible with a known synthesis processes, or that the synthesised molecule is not suitable for certain measurements processes leading to respective technical application properties. Such information can be provided as part of the producability information. Generally, the producability information can refer to categorical information that indicates whether the molecule refers to one or more categories. For example, a category can refer to whether or not the molecule is producible, or whether or not a certain application property can be measured for the molecule. Providing at least some explored molecules with such information in addition to or instead of technical application properties as part of the explored data set, allows to further train the determination model based on these explored molecules to at least note when a molecule might not be suitable for an application, for instance, due to synthesis problems. Generally, for this embodiment it is preferred that more explored molecules are provided with technical application properties than explored molecules provided with producability information instead of technical application properties.

The unexplored data set comprises a plurality of molecule characterizing parameter values for a plurality of molecule characterizing parameters associated with synthesis specifications and/or molecular structures corresponding to a plurality of unexplored molecules. Preferably, the unexplored data set consist of a plurality of molecule characterizing parameter values for a plurality of molecule characterizing parameters associated with synthesis specifications and/or molecular structures corresponding to a plurality of unexplored molecules. Thus, the unexplored data set does not comprise technical application properties associated with the unexplored molecules. This allows for an easy and fast generation of a large amount of unexplored molecules, in particular, molecular structures and/or synthesis specifications and corresponding molecule characterizing parameter values for generating the unexplored data set. For example, in the case of polymers based on a starting synthesis specification, parameters of the synthesis specification can be varied to generate a new synthesis. In the case of small molecules, a data base query of similar molecules based on a fingerprint can be performed. An alternative would be to select a diverse subset of molecules out of a larger set of possible molecules spanning a certain chemical scope. Moreover, also arbitrary variations from a starting synthesis specification and/or molecular structure can be utilized for generating a huge amount of unexplored synthesis specifications and/or molecular structures. Based on these synthesis specifications and/or molecular structures, known methods, for instance, as described above, can then be utilized to determine respective molecule characterizing parameter values for the unexplored molecules associated with respective synthesis specification and/or molecular structures. In this way, huge unexplored data sets can be generated comprising, for instance, thousands of unexplored molecules. In particular, the explored data set and the unexplored data set comprise for each molecule at least some, preferably, for all, molecule characterizing parameter values for the same molecule characterizing parameters. For example, the unexplored data set can be generated such that molecule characterizing parameters are calculated for which respective values are provided in the explored data set. However, also in the explored data set molecule characterizing parameters can be calculated that refer to molecule characterizing parameters provided in the unexplored data set. In particular, the such generated unexplored data set can be utilized to define the application context for the to be trained determination model. In particular, the unexplored data set can be generated taking respective constraints for the intended application of the model into account. Based on the unexplored data set defining the application context, the explored data set can then be utilized for generating a training data set for the determination model. For example, from the explored data set explored molecules and/or molecule characterizing parameters can then be selected to form the training data set. It has been found by the inventors that in this way a training data set can be provided for training a determination model that is specifically tailored to the application context of the technical application property without cumbersome trial-and-error processes and without consuming high computational resources. The such trained determination model then allows for a particularly accurate determination of a technical application property of a molecule falling within the application context of the determination model.

In a further step, the determined technical application property of the potential target molecule is then compared with the target technical application property. Based on the comparison, it is then decided between a) determining the potential target molecule as the target molecule, or b) providing a potential target molecule and repeating the determination of the technical application property utilizing the new potential target molecule. Preferably, step a) is performed if the comparison determines that the determined technical application property of the potential target molecule meets the target technical application property and step b) is performed if the comparison determines that the determined technical application property of the potential target molecule does not meet the target technical application property. In particular, the comparison of the determined technical application property with the target technical application property allows to determine whether the determined technical application property fulfils a predetermined criterion, for example, that the determined technical application property meets the target technical application property within predetermined limits. If such a criterion is fulfilled, the potential target molecule is determined as the target molecule and the method proceeds to the next step. However, if the comparison indicates that the determined technical application property does not meet the target technical application property within the predetermined limits, a next iteration step utilizing a new potential target molecule has to be processed. In particular, for each iteration step of the iteration a new potential target molecule is determined, preferably based on the previous potential target molecule, for instance, by amending one or more features of the previous synthesis specification and/or molecular structure of the previous molecule. However, a new potential target molecule can also be generated by arbitrarily choosing a new potential target synthesis specification and/or molecular structure from a huge amount of previously generated potential target synthesis specifications and/or molecular structures. Moreover, also more sophisticated methods can be utilized for selecting a new potential target molecule from a plurality of previously already generated potential target molecules. Based on the new potential target molecule, in each iteration step again the trained determination model is utilized for determining a technical application property and the such determined technical application property is again compared with the target technical application property such that the comparison can again lead to a further iteration step or if the respective criterion is fulfilled the respective new potential target molecule can be selected as the target molecule. Moreover, also an additional abortion criterion for the iteration can be selected, for instance, a number of iteration steps can be determined before the iteration is aborted with a notification to a user that no target molecule could be found for the respective technical application property. However, alternatively, after a predetermined amount of iteration steps the method can further comprise amending the target technical application property, for instance, by increasing the predetermined limits around the technical application property and to repeat the iteration while utilizing the increased limits during the comparison. This can allow to find a target molecule that meets the technical application property as much as possible, even if a meeting of the original goal might not be possible. After the target molecule has been determined as described above, the target molecule and optionally, further information like a molecular structure or synthesis specification for producing the molecule can be provided, for instance, to a user via an output unit. In the case of a small molecule, a data-driven retro-synthesis planner can be used, which provides potential starting material for the synthesis of the target molecule. Afterwards, a data base can be used, which contains many published synthesis routes. This delivers possible instructions for the synthesis of the target molecule out of the suggested starting materials. Alternatively, a data-driven tool can be used to provide the synthesis instructions as well as the estimated likelihood of a successful synthesis.

In an embodiment, the providing of the determined target molecule comprises generating control signals based on the target molecule and providing the control signals, wherein the control signals are configured to control a production system for producing the target molecule in accordance with the target synthesis specification. Preferably, the control signals are provided to a respective production system configured for producing the target molecule based in the control signals. Generally, the control signals can be provided in any format that allows to directly or indirectly control a production system for producing the target molecule in accordance with the target synthesis specification. For example, the control signals can be provided in a format that allows a production system management application to interpret the control signals and then to control the production system, for example, a synthesis robot, accordingly to produce the target molecule. However, the control signals can also be provided in a format that directly allows a control of the respective production system to produce a target molecule, for instance, that directly allows to control components, for example, to start or stop a heating unit, open or close valves, start or stop a mixer, etc. Generally, the production system can refer to any fully or partly automated production system provided in a laboratory or industrial environment that is generally configured to produce molecules based on synthesis specifications.

In an embodiment, the parameterizing of the machine learning based determination model is based on determining a similarity measure between members of the explored data set and members of the unexplored data set. Generally, in this context a similarity measure can be defined as a measure that determines a distance between one or more members of the explored data set and one or more members of the unexplored data set in a predetermined space. Preferably, the predetermined space is defined by one or more molecule characterizing parameters. Thus, the similarity measure in this case is indicative of how near one or more members of an explored data set are to one or more members of the unexplored data set with respect to one or more molecule characterizing parameters. In this context, a member of the explored data set refers to a molecule and all quantities associated with the molecule provided by the respective explored data set and a member of the unexplored data set refers to a molecule and all quantities associated with the molecule provided by the unexplored data set. In particular, it is preferred that the similarity measure between members of the explored data set and members of the unexplored data set is determined with respect to the molecule characterizing parameters of the members of the explored data set and the members of the unexplored data set. In particular, the similarity measure is indicative of distances between the explored data set and members of the unexplored data set with respect to a subset of the molecule characterizing parameters. Utilizing a similarity measure between members of the explored data set and members of the unexplored data set allows to determine for different molecule characterizing parameters which members of the explored data set are most similar to members of the unexplored data set. This allows to select not only members of the explored data set but also combinations of molecule characterizing parameters that are most suitable for being part of a training data set for training the determination model for the technical application context defined by the unexplored data set.

Preferably, the parameterizing of the machine learning base determination model comprises selecting a subset of the molecule characterizing parameters of the explored data set as training molecule characterizing parameters based on the similarity measure, wherein the parameterization of the machine learning based determination model utilizes a training data set comprising from the explored data set: a) the technical application property of at least two explored molecules of the plurality of explored molecules, and b) molecule characterizing parameter values for the training molecule characterizing parameters associated with synthesis specifications and/or molecular structures corresponding to each of the at least two explored molecules. Generally, the selected subset of the molecule characterizing parameters of the explored data set can refer to a combination of one or more molecule characterizing parameters for which values are provided by the explored data set. In particular, it is preferred that the subset of the molecule characterizing parameters is selected by comparing similarity measures for different combinations of molecule characterizing parameters of the members of the explored data set and members of the unexplored data set and determine from these combinations the combination in which members of the explored data set are most similar to members of the unexplored data set, wherein then the respective combination of molecule characterizing parameters can be selected as subset. Thus, based on the similarity measure it can be determined which combination of molecule characterizing parameters allows an explored data set to optimally cover the technical application context of the determination model defined by the unexplored data set.

Preferably, the subset of the molecule characterizing parameters is selected based on an optimization of the similarity measure with respect to the subset of the molecule characterizing parameters between the members of the unexplored data set and the explored data set. In particular, it is preferred that the optimization refers to determining a subset of molecule characterizing parameters for which the members of the unexplored data set and the members of the explored data set are most similar. Putting this in another way, the similarity measure is optimized such that in a space defined by the subset of molecule characterizing parameters the members of the explored data set cover the part of the space defined by the unexplored data set as good as possible. Thus, training a determination model with such a determined training data set allows to generate a determination model that is in particular well suited for the intended technical application context without the necessity for trial-and-error runs or for performing further experiments to create additionally explored molecules.

Preferably, the similarity measure is mathematically defined for the optimization as

$$\frac{1}{S} \cdot {\sum }_{f , i , j} {c}_{f} \cdot D ( {X}_{i , f}^{scr} , {X}_{j , f}^{tr} )$$

wherein

$${X}_{i , f}^{scr} and {X}_{j , f}^{tr}$$

are molecule characterizing parameters from the unexplored (scr) and the explored (tr) data set, where f refers to a specific characterizing parameter and i, j to a respective molecule of the unexplored and explored data sets, respectively, the coefficient c_{f} can be 0 or 1 depending on whether a respective characterizing parameter is selected and the sum over f is equal to K, the number of characterizing parameters in a selected subset of the characterizing parameters, D is a generic distance function between any given two data points in a space defined by the selected subset of characterizing parameters, and S is the number of data points in the unexplored data set. The number of characterizing parameters in a selected subset of the characterizing parameters K can refer to a predetermined number, for instance, it might be known from experience that less than three characterizing parameters might lead to less accurate results when determining a technical application property with a such trained determination model, whereas it might be not advantageous to have to provide more than 20 characterizing parameters to the determination model to determine a technical application property. For instance, a high number of characterizing parameters that have to be provided to the determination model for determining a technical application property might lead to an overfitting of the model that should be avoided, since it can lead to a decreased predictivity of the resulting model on datasets different to that used for training of the model. Thus, for example, the number of characterizing parameters for a subset of the characterizing parameters can be predetermined based on such considerations as outlined above. However, it is preferred that the number of characterizing parameters in a selected subset of the characterizing parameters is itself subject to an optimization process and can thus be regarded as a hyperparameter. In particular, the optimization of the similarity measure can also include an optimization over the number of characterizing parameters in a selected subset of the characterizing parameters such that this number does not have to be predetermined or fixed during the optimization process.

In a preferred embodiment, the subset is selected by further optimizing over a determination accuracy of the determination model with respect to the subset of molecule characterizing parameters. Selecting the subset of characterizing parameters further based on an optimization over a determination accuracy of the determination model with respect to the subset of molecule characterizing parameters allows to take a potential accuracy of the determination model also already into account during the determination of the training data set. This allows to optimize the selection of the characterizing parameters in the training data set also with respect to the determination accuracy already during the determination of the training data set. Preferably, the determination accuracy is mathematically defined for the optimization as

$$λ \cdot \frac{1}{T} {\sum }_{j} D * ( F ( {X}_{j , f}^{tr} ) , {Y}_{j}^{tr} )$$

Wherein

$$F ( {X}_{j , f}^{tr} )$$

refers to the technical application properties determined with the determination model that is trained with a respective training data set based on a respective selected subset,

$${Y}_{j}^{tr}$$

refers to the technical application properties for a respective molecule, T is the number of data points in the explored data set, D* is a generic distance measured between the technical application properties determined for the molecules of the explored data set with the determination model that is trained and the technical application properties of the molecules of the explored data set, and the parameter λ refers to a weighting of the determination accuracy with respect to other terms of the optimization.

In a preferred embodiment, the subset is selected by further optimizing over an applicability of the determination model in the unexplored data set. In particular, the applicability of the determination model in the unexplored data set can be measured by utilizing a criterion that determines the coverage of a space defined by the unexplored data set by the explored data set with respect to the selected subset of molecule characterizing parameters. For example, if the explored data set only covers a very small part of the space defined by the unexplored data set, i.e. if the coverage is incomplete, the determination model might not be applicable to the complete intended technical application context defined by the unexplored data set. Thus, also taking the applicability of the determination model in the unexplored data set into account, for instance, by utilizing a coverage measure, the training data set can directly also be optimized with respect to the applicability and an intended applicability of the determination model can be ensured as much as possible. Preferably, the applicability is mathematically defined for the optimization as

$$μ \cdot \frac{1}{S} \cdot {\sum }_{i} G ( F ( {X}_{i , f}^{scr} ) , {Y}_{j}^{tr} )$$

Wherein

$$F ( {X}_{i , f}^{scr} )$$

refers to the technical application properties determined with the determination model that is trained,

$${Y}_{j}^{tr}$$

refers to the technical application properties for a respective molecule, G is a function that evaluates the applicability of the determination model in the unexplored data set, for example, an Euclidean distance, S is the number of data points in the unexplored data set, and the parameter μ refers to a weighting of a measure of applicability with respect to other terms of the optimization. Generally, the weighting parameters μ and λ that weigh the additional measures with respect to the similarity measure during the optimization can refer to predetermined values that are determined, for instance, based on user experience, theoretical considerations or a resampling method. For example, an increase of μ increases an application space for the determination model around the explored data set, wherein a decrease also decreases the application space of the determination model. Δ determines strongly whether the determination model follows the explored data set. However, these parameters can also be regarded as hyperparameters that can also be subjected to the optimization, i.e. can be varied during the optimization.

In an embodiment, the selecting of the subset of molecule characterizing parameters comprises a) determining a parameter training space based on the unexplored data set, wherein the parameter training space is defined by the molecule characterizing parameter values of the molecule characterizing parameters of the plurality of unexplored molecules, b) determining a subspace of the parameter training space such that the molecule characterizing parameter values of the molecule characterizing parameters of the explored data set cover the subspace in accordance with a predetermined criterion based on the similarity measure, and c) selecting the subset of molecule characterizing parameters based on the determined subspace. Generally, the predetermined criterion can additionally also be based on the other measures already defined above. Accordingly, optimizing the similarity measure and optionally also one or more of the above defined measures with respect to the molecule characterizing parameters refers also to optimizing the subspace of the parameter training space such that the explored data set covers the subspace as good as possible.

In an embodiment, the providing of the unexplored data set comprises a) receiving information indicative of constraints for generating a molecule, and b) generating the unexplored data set based on the received information. The constraints can refer to any constraints for the generating of a molecule, for instance, to constraints provided by technical constraints of a production system for generating a molecule to production constraints referring, for example, to constraints in an availability of starting substances or catalysts or other necessary substances for generating a molecule, to physical or chemical constraints that do not allow to generate a specific molecule. Such constraints can be applicable to generally all molecules, for instance, if they refer to physical or chemical constraints and thus can generally be taken into account when generating an unexplored data set or can refer to specific constraints, for example, to the constraints of technical facilities of a potential user that have to be taken into account when generating the unexplored data set. For example, if technical constraints indicate that the production facilities of a potential user only allow the production of molecules within a certain temperature range and only based on certain starting substances, the synthesis specifications of the unexplored data set can easily be generated taking these constraints into account such that only molecules are considered in the unexplored data set that can be generated by the respective production facilities of a potential user. In a preferred embodiment the constraints cause to constrain the potential target molecules to molecules for which a respective synthesis specification is known or determinable. Generally, the above mentioned constraints allows also to determine a training data set for the determination model such that the determination model is specifically trained for this intended application, i.e. is trained only for molecules that can be produced by the production facilities. Moreover, it is preferred that during the determination of a target molecule the new potential target molecules are generated also taking the respective constraints into account such that only potential target molecules are considered during the iteration that can actually be produced by the respective target facilities of a potential user.

In an embodiment, the parameterizing of the machine learning base determination model comprises selecting a subset of molecule characterizing parameters from the molecule characterizing parameters of the explored data set based on the explored data set and the unexplored data set as training molecule characterizing parameters, wherein the parameterization of the machine learning based determination model utilizes a training data set comprising from the explored data set a) the technical application property for at least two explored molecules of the plurality of explored molecules, and b) molecule characterizing parameter values for the training molecule characterizing parameters associated with synthesis specification and/or molecular structures corresponding to each of the at least two explored molecules. Preferably, the selecting of the subset comprises a) determining a parameter training space based on the unexplored data set, wherein the parameter training space is defined by the molecule characterizing parameter values of the molecule characterizing parameters of the plurality of unexplored molecules, b) determining a subspace of the parameter training space such that the molecule characterizing parameter values of the molecule characterizing parameters of the explored data set cover the subspace in accordance with a predetermined criterion, and c) selecting the training molecule characterizing parameters based on the determined subspace. Further, it is preferred that the predetermined criterion refers to a similarity between members of the explored data set and members of the unexplored data set. In particular, the similarity can be determined utilizing the similarity measure as already described above. Moreover, the predetermined criterion can also refer additionally to the other measures already defined above and determining the respective subspace can refer to an optimization of the similarity measure and optionally additionally of any of the above already described other measurements.

In a further aspect of the invention, a computer implemented method for generating a machine learning based determination model such that the trained determination model is adapted to determine a technical application property of a molecule based on one or more molecule characterizing parameters is presented, wherein a molecule characterizing parameter is associated with a synthesis specification and/or molecular structure corresponding to a molecule, wherein the method comprises i) providing an explored data set comprising a) a technical application property for a plurality of explored molecules and b) a plurality of molecule characterizing parameter values for a plurality of molecule characterizing parameters associated with synthesis specification and/or molecular structures corresponding to each of the plurality of explored molecules, ii) providing an unexplored data set comprising a plurality of molecule characterizing parameter values for a plurality of molecule characterizing parameters associated with synthesis specification and/or molecular structures corresponding to a plurality of unexplored molecules, iii) generating the trained determination model by parameterizing the machine learning based determination model based on the explored data set and the unexplored data set such that the trained determination model is adapted to determine a technical application property of a molecule based on one or more molecule characterizing parameters of the molecule, iv) providing the generated trained determination model. Preferably, the providing of the unexplored data set comprises a generation of the unexplored data set based on an intended application of the determination model. In particular, as already described above, a plurality of synthesis specifications and/or molecular structures can be generated based on the intended application, in particular, based on constraints.

In a further aspect, a computer implemented method for determining on one or more molecule characterizing parameters for training a machine learning based determination model such that the trained determination model is adapted to determine a technical application property of a molecule based on the one or more molecule characterizing parameters is presented, wherein a molecule characterizing parameter is associated with a synthesis specification and/or molecular structure corresponding to the molecule, wherein the method comprises i) providing an explored data set comprising a) a technical application property for a plurality of explored molecules and b) a plurality of molecule characterizing parameter values for a plurality of molecule characterizing parameters associated with synthesis specification and/or molecular structures corresponding to each of the plurality of explored molecules, ii) providing an unexplored data set comprising a plurality of molecule characterizing parameter values for a plurality of molecule characterizing parameters associated with synthesis specification and/or molecular structures corresponding to a plurality of unexplored molecules, iii) selecting on or more molecule characterizing parameters from characterizing parameters of the explored data set based on the explored data set and the unexplored data set, and iv) providing the selected molecule characterizing parameter. In an embodiment, it is preferred that the providing of the unexplored data set comprises a generation of the unexplored data set based on an intended application of the determination model. In particular, as already described above, a plurality of synthesis specifications and/or molecular structures can be generated based on the intended application, in particular, based on constraints.

Generally, the same embodiments and definitions described with respect to the method for determining a target molecule can also be applied to respective features of the methods for determining a determination model and for determining the molecule characterizing parameters for training the determination model.

In a further aspect, an interface method for providing a determination model is presented, wherein the interface comprises i) receiving, via an input unit, an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules ii) receiving, via an input unit, information indicative of an unexplored data set comprising a plurality of molecule characterizing parameter values for a plurality of molecule characterizing parameters associated with synthesis specifications and/or molecular structures corresponding to a plurality of unexplored molecules, iii) interfacing, via an interface unit, with a processor performing the method as described above for providing the explored data set and the information, and receiving the trained determination model, and iv) providing, via an output unit, the trained determination model. In an embodiment the interface method can comprise the steps performed by the processor according to the method as described above for providing the explored data set and the information.

In a further aspect, an interface method for providing one or more molecule characterizing parameters for training a machine learning based determination model is presented, wherein the interface comprises i) receiving, via an input unit, an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules, ii) receiving, via an input unit, information indicative of an unexplored data set comprising a plurality of molecule characterizing parameter values for a plurality of molecule characterizing parameters associated with synthesis specifications and/or molecular structures corresponding to a plurality of unexplored molecules, iii) interfacing, via an interface unit, with a processor performing the method as described above for providing the explored data set and the information, and receiving the determined molecule characterizing parameters for training a machine learning based determination model, and iv) providing, via an output unit, the determined molecule characterizing parameters. In an embodiment the interface method can comprise the steps performed by the processor according to the method as described above for providing the explored data set and the information, and receiving the determined molecule characterizing parameters for training a machine learning based determination model.

In a further aspect, an interface method for providing a target synthesis specification indicative of a target molecule comprising a target technical application property is presented, wherein the interface comprises i) receiving, via an input unit, a target technical application property, ii) interfacing, via an interface unit, with a processor performing the method as described above for providing the target technical application property, and receiving the determined target synthesis specification, and iii) providing, via an output unit, the determined target synthesis specification. In an embodiment the interface method can comprise the steps performed by the processor according to the method as described above for providing the target technical application property, and receiving the determined target synthesis specification.

In a further aspect, an apparatus for generating a machine learning based determination model such that the trained determination model is adapted to determine a technical application property of a molecule based on one or more molecule characterizing parameters is presented, wherein a molecule characterizing parameter is associated with a synthesis specification and/or molecular structure corresponding to the molecule, wherein the apparatus comprises i) an input interface configured to provide an explored data set comprising a) a technical application property for a plurality of explored molecules and b) a plurality of molecule characterizing parameter values for a plurality of molecule characterizing parameters for each of the plurality of explored molecules, and to provide an unexplored data set comprising a plurality of molecule characterizing parameter values for a plurality of molecule characterizing parameters for a plurality of unexplored molecules, ii) a processor configured to generate the trained determination model by parameterizing the machine learning based determination model based on the explored data set and the unexplored data set such that the trained determination model is adapted to determine a technical application property of a molecule based on one or more molecule characterizing parameters of the molecule, and iii) an output interface configured to provide the generated trained determination model.

In a further aspect, an apparatus for determining on one or more molecule characterizing parameters for training a machine learning based determination model such that the trained determination model is adapted to determine a technical application property of a molecule based on the one or more molecule characterizing parameters is presented, wherein a molecule characterizing parameter is indicative of a characterizing characteristic of a molecule and/or is derivable from one or more characterizing characteristics of the molecule, wherein the apparatus comprises i) a input interface configured to provide an explored data set comprising a) a technical application property for a plurality of explored molecules and b) a plurality of molecule characterizing parameter values for a plurality of molecule characterizing parameters for each of the plurality of explored molecules, and to provide an unexplored data set comprising a plurality of molecule characterizing parameter values for a plurality of molecule characterizing parameters for a plurality of unexplored molecules, ii) a processor configured to select on or more molecule characterizing parameters from characterizing parameters of the explored data set based on the explored data set and the unexplored data set, and iii) an output interface configured to provide the selected molecule characterizing parameter.

In a further aspect, an apparatus for determining a target synthesis specification indicative of a target molecule comprising a target technical application property is presented, wherein the apparatus comprises i) an input interface configured to provide a target technical application property, and to provide a digital representation of a potential target molecule indicative of or associated with characterizing parameters of a potential target molecule, ii) a processor configured to utilize a trained determination model for determining a technical application property of the molecule, wherein the trained determination model has been parameterized based on an explored data set and an unexplored data set such that the trained determination model is adapted to determine a technical application property of a molecule based on one or more molecule characterizing parameters of the molecule, wherein the explored data set comprises a) a technical application property for a plurality of explored molecules and b) a plurality of molecule characterizing parameter values for a plurality of molecule characterizing parameters associated with synthesis specifications and/or molecular structures corresponding to each of the plurality of explored molecules, and the unexplored data set comprising a plurality of molecule characterizing parameter values for a plurality of molecule characterizing parameters associated with synthesis specifications and/or molecular structures corresponding to a plurality of unexplored molecules, and compare the determined technical application property of the potential target molecule with the target technical application property and, based on the comparison, either a) determining the potential target molecule as the target molecule, or b) providing a new potential target molecule and repeating the determination of the technical application property utilizing the new potential target molecule, and iii) an output interface configured to provide the determined target molecule and the corresponding determined target technical application property.

In a further aspect, an interface apparatus for providing a determination model is presented, wherein the interface apparatus comprises i) an input unit configured to receive an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules, and receive information indicative of an unexplored data set comprising a plurality of molecule characterizing parameter values for a plurality of molecule characterizing parameters associated with synthesis specifications and/or molecular structures corresponding to a plurality of unexplored molecules, ii) an interface unit configured to interface with a processor performing the method as described above for providing the explored data set and the information, and receiving the trained determination model, and iii) an output unit configured to provide the trained determination model. In an embodiment the interface apparatus can comprise the processor performing the method as described above for providing the explored data set and the information.

In a further aspect, an interface apparatus for providing one or more molecule characterizing parameters for training a machine learning based determination model is presented, wherein the interface apparatus comprises i) an input unit configured to receive an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules and information indicative of an unexplored data set comprising a plurality of molecule characterizing parameter values for a plurality of molecule characterizing parameters associated with synthesis specifications and/or molecular structures corresponding to a plurality of unexplored molecules, ii) an interface unit configured to interface with a processor performing the method as described above for providing the explored data set and the information, and receiving the determined molecule characterizing parameters for training a machine learning based determination model, and iii) an output unit configured to provide the determined molecule characterizing parameters. In an embodiment the interface apparatus can comprise the processor performing the method as described above for providing the explored data set and the information, and receiving the determined molecule characterizing parameters for training a machine learning based determination model.

In a further aspect, an interface apparatus for providing a target molecule comprising a target technical application property is presented, wherein the interface apparatus comprises i) an input unit configured to receive a target technical application property, ii) an interface unit configured to interface with a processor performing the method as described above for providing the target technical application property, and receiving the determined target synthesis specification, and iii) an output unit configured to provide the determined target molecule. In an embodiment the interface apparatus can comprise the processor performing the method as described above for providing the target technical application property, and receiving the determined target synthesis specification.

In a further aspect, a use of determined training molecule characterizing parameters for training a machine learning based determination model is presented, wherein the training molecule characterizing parameters have been generated utilizing the apparatus and method as described above.

In a further aspect, a computer program product for determining a target molecule comprising a target technical application property is presented, wherein the computer program product comprises program code means for causing a computing system to execute a method as described above.

In a further aspect, control signals generated utilizing a method as described above is presented.

In a further aspect, a use of control signals generated utilizing a method as described above for controlling a producing system, in particular, laboratory equipment, for producing a target molecule.

In a further aspect, a trained determination model generated utilizing a method as described above is presented.

In a further aspect, a data set comprising characterizing parameters is presented, wherein the characterizing parameters are determined utilizing a method as described above.

In a further aspect of the invention an apparatus for optimizing a training data set for training a machine learning based determination model such that the trained determination model is adapted to determine a technical application property of a molecule based on one or more molecule physicochemical parameters is presented, wherein a molecule physicochemical parameter is indicative of a physicochemical characteristic of a molecule and/or is derivable from one or more physicochemical characteristics of the molecule, wherein the apparatus comprises i) an explored data providing unit for providing an explored data set comprising a) a property for a plurality of molecules and b) a plurality of molecule physicochemical parameter values for a plurality of molecule physicochemical parameters for the plurality of molecules, ii) a parameter training space providing unit for providing a parameter training space, wherein the parameter training space is defined in a molecule physicochemical parameter space with respect to an intended application of the determination model, iii) a subspace determination unit for determining a subspace of the parameter training space defined by one or more of the molecule physicochemical parameters defining the parameter training space, wherein the subspace is determined such that the training data set covers the subspace in accordance with a predetermined criterion, and iv) a training molecule determination unit for determining training molecule physicochemical parameters for training the determination model based on the determined molecule physicochemical parameters defining the determined subspace, and v) a training data selection unit for selecting data from the explored data set such that the training data set comprises a property of a plurality of molecules, and molecule physicochemical parameter values for the determined training molecule physicochemical parameters for the plurality of molecules. In particular, it is preferred that the parameter training space providing unit is adapted to determine the parameter training space based on an unexplored data set as defined above. Preferably, the predetermined criterion refers to a similarity between the explored data set and the unexplored data set and the subspace is determined by optimizing a similarity measure between the explored data set and the unexplored molecule data set with respect to the molecule physicochemical parameters. Generally, also for this aspect the same embodiments and definitions as already described above can be applied.

In a preferred embodiment the molecule is a polymer. In this embodiment a computer implemented method for determining a target synthesis specification indicative of a target polymer comprising a target technical application property is presented, wherein the method comprises i) providing a target technical application property, ii) providing a digital representation of a potential target synthesis specification indicative of or associated with characterizing parameters of a potential target polymer, iii) utilizing a trained determination model for determining a technical application property of the potential target polymer, wherein the trained determination model has been parameterized based on an explored data set and an unexplored data set such that the trained determination model is adapted to determine a technical application property of a polymer based on one or more polymer characterizing parameters of the polymer, wherein the explored data set comprises a) a technical application property for a plurality of explored polymers and b) a plurality of polymer characterizing parameter values for a plurality of polymer characterizing parameters associated with synthesis specifications corresponding to each of the plurality of explored polymers, and the unexplored data set comprising a plurality of polymer characterizing parameter values for a plurality of polymer characterizing parameters associated with synthesis specifications corresponding to a plurality of unexplored polymers, iv) comparing the determined technical application property of the potential target polymer with the target technical application property and, based on the comparison, either a) determining the potential target polymer as the target polymer and the potential target synthesis specification as the target synthesis specification, or b) providing a new potential target synthesis specification of a new potential target polymer and repeating the determination of the technical application property utilizing the new potential target synthesis specification of the new potential target polymer, v) providing the determined target polymer and the target synthesis specification. Preferably, the providing of the determined target polymer and the target synthesis specification comprises generating control signals based on the target synthesis specification and providing the control signals, wherein the control signals are configured to control a production system for producing the target polymer in accordance with the target synthesis specification. Moreover, a computer implemented method for generating a machine learning based determination model such that the trained determination model is adapted to determine a technical application property of a polymer based on one or more polymer characterizing parameters is presented, wherein a polymer characterizing parameter is associated with a synthesis specification corresponding to a polymer, wherein the method comprises i) providing an explored data set comprising a) a technical application property for a plurality of explored polymers and b) a plurality of polymer characterizing parameter values for a plurality of polymer characterizing parameters associated with synthesis specifications corresponding to each of the plurality of explored polymers, ii) providing an unexplored data set comprising a plurality of polymer characterizing parameter values for a plurality of polymer characterizing parameters associated with synthesis specifications corresponding to a plurality of unexplored polymers, iii) generating the trained determination model by parameterizing the machine learning based determination model based on the explored data set and the unexplored data set such that the trained determination model is adapted to determine a technical application property of a polymer based on one or more polymer characterizing parameters of the polymer, iv) providing the generated trained determination model. Further, a computer implemented method for determining on one or more polymer characterizing parameters for training a machine learning based determination model such that the trained determination model is adapted to determine a technical application property of a polymer based on the one or more polymer characterizing parameters is presented, wherein a polymer characterizing parameter is associated with a synthesis specification corresponding to the polymer, wherein the method comprises i) providing an explored data set comprising a) a technical application property for a plurality of explored polymers and b) a plurality of polymer characterizing parameter values for a plurality of polymer characterizing parameters associated with synthesis specifications corresponding to each of the plurality of explored polymers, ii) providing an unexplored data set comprising a plurality of polymer characterizing parameter values for a plurality of polymer characterizing parameters associated with synthesis specifications corresponding to a plurality of unexplored polymers, iii) selecting on or more polymer characterizing parameters from characterizing parameters of the explored data set based on the explored data set and the unexplored data set, and iv) providing the selected polymer characterizing parameter. Moreover, an interface method for providing a target synthesis specification indicative of a target polymer comprising a target technical application property is presented, wherein the interface comprises i) receiving, via an input unit, a target technical application property, ii) interfacing, via an interface unit, with a processor performing the method according to any of claims 1 to 8 for providing the target technical application property, and receiving the determined target synthesis specification, and ii) providing, via an output unit, the determined target synthesis specification. Also, an apparatus for determining a target synthesis specification indicative of a target polymer comprising a target technical application property is presented, wherein the apparatus comprises i) an input interface configured to a) provide a target technical application property, and b) provide a digital representation of a potential target synthesis specification indicative of or associated with characterizing parameters of a potential target polymer, ii) a processor configured to a) utilize a trained determination model for determining a technical application property of the polymer, wherein the trained determination model has been parameterized based on an explored data set and an unexplored data set such that the trained determination model is adapted to determine a technical application property of a polymer based on one or more polymer characterizing parameters of the polymer, wherein the explored data set comprises I) a technical application property for a plurality of explored polymers and II) a plurality of polymer characterizing parameter values for a plurality of polymer characterizing parameters associated with synthesis specifications corresponding to each of the plurality of explored polymers, and the unexplored data set comprising a plurality of polymer characterizing parameter values for a plurality of polymer characterizing parameters associated with synthesis specifications corresponding to a plurality of unexplored polymers, and b) compare the determined technical application property of the potential target polymer with the target technical application property and, based on the comparison, either I) determining the potential target polymer as the target polymer and the potential target synthesis specification as the target synthesis specification, or II) providing a new potential target synthesis specification of a potential target polymer and repeating the determination of the technical application property utilizing the new potential target synthesis specification of the potential target polymer, and iii) output interface configured to provide the determined target polymer and the corresponding determined target technical application property. Further, a com-puter program product for determining a target synthesis specification indicative of a target polymer comprising a target technical application property is presented, wherein the com-puter program product comprises program code means for causing a computing system to execute the method as described above. Moreover, a use of control signals is presented generated utilizing the method described above for controlling a producing system, in particular, laboratory equipment, for producing the target polymer in accordance with the target synthesis specification. It shall be understood that the methods as described above, the apparatuses as described above and the computer program products as described above have similar and/or identical preferred embodiments, in particular, as defined in the dependent claims.

It shall be understood that a preferred embodiment of the present invention can also be any combination of the dependent claims or above embodiments with the respective inde-pendent claim.

These and other aspects of the present invention will be apparent from and elucidated with reference to the embodiments described hereinafter.

### BRIEF DESCRIPTION OF THE DRAWINGS

FIG. 1 shows schematically and exemplarily a method for determining a target synthesis specification indicative of a target molecule comprising a target technical application property,

FIG. 2 shows schematically and exemplarily a method for providing a model for determining a technical application property usable in the method for determining a target synthesis specification,

FIG. 3 shows schematically and exemplarily a principle of the invention,

FIGS. 4 to 6 show schematically and exemplary flowcharts of exemplary embodiments of the invention, and

FIGS. 7 and 8 show schematically and exemplarily block diagrams of exemplary system architectures of systems utilizing the invention.

### DETAILED DESCRIPTION OF THE DRAWINGS

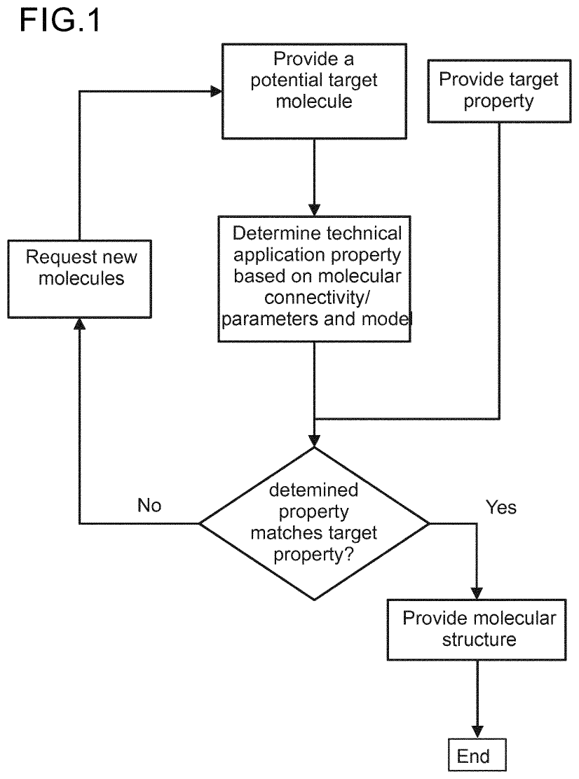

FIG. 1 shows schematically and exemplarily a computer implemented method for determining a target synthesis specification indicative of a target molecule comprising a target technical application property. The method comprises providing a target technical application property that can refer to any technical application property that should be met by a respective target molecule. For example, the target technical application property can be provided in form of a target value together with predetermined limits within which the target molecule should meet the target value. However, the target technical application property can also be provided in form of a target value range, wherein in this case a target molecule should comprise a technical application property value that falls within the target range. Moreover, the target technical application property can also be provided in form of upper or lower limits such that a target molecule should comprise a target technical application property value that lies below or above, respectively, the respective upper or lower limit.

Further, the method comprises providing a digital representation of a potential target molecule indicative of or associated with characterizing parameters of a potential target molecule. The digital representation can refer to any representation that allows to derive the potential target molecule from the digital representation, in particular, the digital representation can be indicative of a chemical structure, i.e. molecular structure, or synthesis specification of the molecule. The characterizing parameters of a potential target molecule can refer to any kind of parameter that allows to characterize, in particular, characteristics of the potential target molecule. For example, the characterizing parameters can comprise process parameters characterizing at least a part of a process in which the potential target molecule can be produced. Further, the characterizing parameters can also comprise recipe parameters characterizing a recipe from which the potential target molecule can be produced, for instance, characterizing substances utilized during the production process, like starting substances or catalysts. Preferably, the characterizing parameters comprise physicochemical parameters of the potential target molecule itself, for example, molecule descriptors like quantum mechanical descriptors. Preferably, the characterizing parameters are indicative of a molecular connectivity or other properties of the molecule. The digital representation of the potential target molecule is indicative of or associated with the characterizing parameters of the potential target molecule. For example, the characterizing parameters of a potential target molecule can be derived from a molecular structure and/or synthesis specification provided as digital representation. However, at least some of the characterizing parameters can also be already part of the digital representation of the potential target molecule or can be available on respective databases, when the potential target molecule is known. If necessary, generally known methods for deriving the characterizing parameters from the target molecule, for example, based on a molecular structure and/or synthesis specification, can be utilized, like 3D simulations, quantum mechanical simulations, electron structure calculations, etc. However, characterizing parameters can also be directly read from the potential target molecule, for instance, in case of the characterizing parameters referring to molecular connectivity or number of atoms that are generally provided as part of a molecular identification. Moreover, suitable characterizing parameters can also be already stored for a plurality of potential target molecules and can then be provided by accessing the respective storage and selecting the characterizing parameters associated with the specific potential molecule.

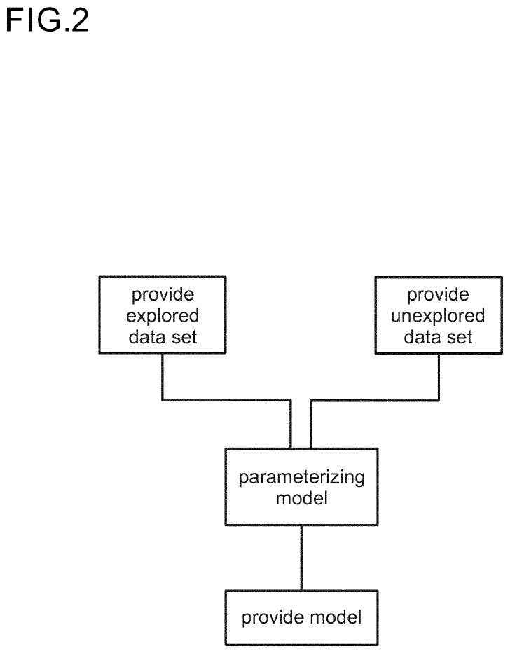

In a next step, a determination model is utilized for determining a technical application property of the potential target molecule, wherein the trained determination model has been parameterized based on an explored data set and an unexplored data set such that the trained determination model is adapted to determine a technical application property of a molecule based on one or more molecule characterizing parameters of the molecule. Further details referring to the training, in particular, parameterizing of the determination model, will be described with respect to FIG. 2. Generally, a determination model is utilized that is suitable for the intended application, i.e. that has been trained to determine a technical application property referring to the target technical application property based on the derived or provided characterizing parameters and is generally applicable to the potential target molecule. Thus, generally for different molecule types, different to be determined technical application properties and/or different characterizing parameters different determination models can be stored, for instance, on a storage unit, and can then be utilized to determine the technical application property. The determination model is a data-driven model parameterized such that it can determine based on the digital representation, for example, based on molecule descriptors indicative of the physicochemical characteristics of subgroups of the molecule being a polymer, the technical application property associated with the molecule. In a preferred embodiment, the data-driven model refers to a machine learning model, for instance, regression model based algorithms or a classifier model based algorithms. A regression model based algorithm can be based on any of a neural network algorithm, a LASSO algorithm, a Ridge Regression algorithm, a MARS algorithm, and a Random Forest algorithm. A classifier based model algorithm can be based on any of a Random Forest algorithm and a SVM algorithm. The inventors have found that for most applications, in particular, Random Forest and MARS based algorithms are suitable. Based on the characterizing parameters associated with or indicated by the potential target polymer and the determination model, a respective technical application property of the potential target molecule is determined.

In a further step, the determined technical application property and the target technical application property are compared. In case the determined technical application property of the potential target molecule does not meet the target technical application property, a new potential target molecule is provided, in particular, comprising a new molecular structure and/or synthesis specification. For example, the new potential target molecule can be provided from a storage on which a plurality of potential target molecules are already stored. A new potential target molecule can then be selected from the already stored plurality of potential target molecules arbitrarily, or based on predetermined rules. However, a new potential target molecule, in particular, in form of a new potential target synthesis specification and/or molecular structure, can also be generated, for example, by amending one or more of the parameters of the previously utilized potential target synthesis specification and/or molecular structure based on predetermined rules. The determination of the technical application property utilizing the determination model is then repeated with the new potential target molecule. If the determined technical application property meets the target technical application property within predetermined limits, the potential target molecule is determined as the target molecule and the such determined target molecule is provided, for example, in form of an executable control file that can be utilized to control a synthesis process, for instance, in a laboratory, for synthesizing the target molecule.

FIG. 2 shows schematically and exemplarily a computer implemented method for providing a determination model that can be utilized in the computer implemented method described above. In particular, the provided determination model is parameterized based on an explored data set and an unexplored data set. The method comprises providing an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules. Further, an unexplored data set is provided comprising a plurality of molecule characterizing parameter values for a plurality of molecule characterizing parameters associated with synthesis specifications and/or molecular structures corresponding to a plurality of unexplored molecules. In particular, at least some of the molecule characterizing parameters for which characterizing parameter values are provided in the unexplored data set and the explored data set refer to the same molecule characterizing parameters. The such provided explored data set and unexplored data set are then utilized to generate a trained determination model by parameterizing the machine learning based determination model based on the explored data set and the unexplored data set such that the trained determination model is adapted to determine a technical application property of a molecule based on one or more molecule characterizing parameters of the molecule. Generally, it has been found by the inventors that parameterizing a determination model based on an explored data set and an unexplored data set does not only allow for a more effective, i.e. less computational resources and less time consuming, training process of the determination model, but further to a better adap-tion of the trained determination model to an intended application context and thus to more accurate results when utilizing the trained determination model for determining a technical application property of a molecule.

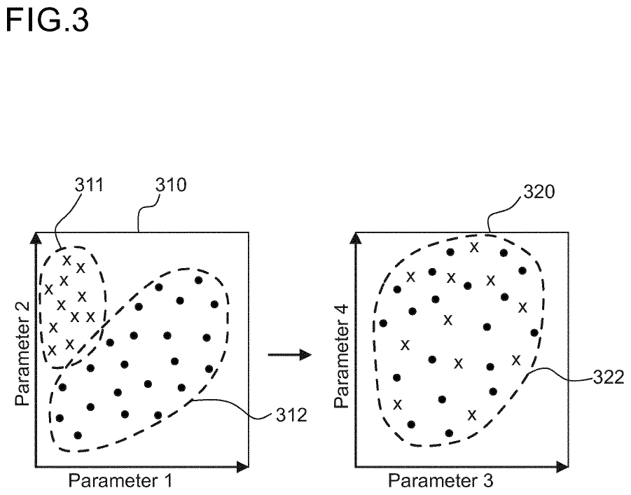

In particular, it is preferred that the explored data set and the unexplored data set are utilized to determine a training data set for training the determination model that is specifically adapted to the application context in which the determination models should be utilized. FIG. 3 shows exemplarily and schematically a background on the determination of a training data set based on the unexplored data set and the explored data set. In particular, in FIG. 3 shows two different cross sections, i.e. subspaces, of a space spanned by all characterizing parameters provided by the explored and unexplored data set. In this schematic example, a cross section 310 of this space spanned by characterizing parameter 1 and characterizing parameter 2 is shown. Further, a cross section 320 spanned by characterizing parameter 3 and characterizing parameter 4 is shown. Based on the values provided for the respective characterizing parameters in the unexplored data set and the explored data set members of the unexplored and explored data set can be recorded in the respective cross sections 310, 320. In particular, each point or cross shown in the respective cross sections 310, 320 can be regarded as being associated with one molecule of the unexplored or explored data set. In the examples shown here, the crosses refer to the explored data set and the points refer to the unexplored data set. Generally, the explored data set refers to molecules for which at least one technical application property is already known, for instance, measured, and thus defines a data set that can generally be utilized for training a determination model due to the already known technical application property. As is exemplarily shown in FIG. 3, in cross section 310 based on the characterizing parameters 1 and 2, the explored molecules, i.e. the members of the explored data set, can only be found in a specific area 311 of the cross section 310, i.e. subspace, for this combination of characterizing parameters. The unexplored data set generally refers to molecules, in particular, to molecular structures and/or synthesis specifications, that are artificially generated in a huge amount, for instance, more than a thousand unexplored molecules, in particular, molecular structures and/or synthesis specifications, can be generated to form the unexplored data set. The unexplored data set is then utilized to define the technical application context intended for the determination model. For example, the unexplored data set can be generated to comprise molecular structures and/or synthesis specifications and associated molecules taking into account specific production constraints or application constraints. Thus, it is preferred that the training data set for training the determination model covers a space defined by the members of the unexplored data set in accordance with a predetermined criterion. As exemplarily shown in FIG. 3 for the first cross section 310, i.e. for the first subspace of the characterizing parameter space defined by the characterizing parameters, the members of the explored data set and the members of the unexplored data set do mainly not cover the same area, i.e. the explored data set covers the area 311 and the unexplored data set covers the area 312. Generally, the predetermined criterion would for such a case determine that the coverage is not well enough. In contrast thereto, in cross section 320, i.e. in a subspace of the characterizing parameter space spanned by characterizing parameters 3 and 4, it is shown that area 322 defined by the unexplored data set is also well covered by the members of the explored data set. Thus, changing a perspec-tive, i.e. selecting a respective subspace of the characterizing parameter space allows to find characterizing parameters, in particular, combinations of characterizing parameters, for which the explored data set is similar to the unexplored data set and thus is very suitable for the intended technical application of the determination model. Thus, for the example shown in FIG. 3 a training data set can be selected that comprises at least two technical application properties for two of the explored molecules of the explored data set and further the values of the characterizing parameters 3 and 4 of the respective explored molecules.

According to the principles explained above with respect to FIG. 3, the unexplored data set and the explored data set are utilized for selecting a training data set for parameterizing the determination model. In particular, the selecting refers to a selecting of the characterizing parameters that are utilized in the training data set for training the determination model. Preferably, the characterizing parameters, i.e. the respective subset of the characterizing parameters, is selected based on a similarity measure indicative of a similarity between members of the explored data set and members of the unexplored data set with respect to a respective subset of the characterizing parameters. One possibility to mathematically find an optimal subset of characterizing parameters such that the members of the explored data set are as similar as possible to the members of the unexplored data set refers to defining an optimization function. For example, a possibility of an optimization function can be defined as

$$O ( {X}_{i , f}^{scr} , {X}_{j , f}^{tr} ) = \frac{1}{S} \cdot {\sum }_{f , i , j} {c}_{f} \cdot D ( {X}_{i , f}^{scr} , {X}_{j , f}^{tr} )$$

wherein

$${X}_{i , f}^{scr} and {X}_{j , f}^{tr}$$

are the molecule characterizing parameters from the unexplored (scr) and the explored (tr) data set, where f refers to a specific characterizing parameter and i, j to a respective molecule of the unexplored and explored data sets, respectively, the coefficient c_{f} can be 0 or 1 depending on whether a respective characterizing parameter is selected and the sum over f is equal to K the number of characterizing parameters in a selected subset of the characterizing parameters, D is a generic distance function between any given data point in a space defined by the selected subset of characterizing parameters, and S is the number of data points in the unexplored data set. The respective function can then be optimized with respect to the selected subset of characterizing parameters such that the members of the explored data set are as similar as possible to the members of the unexplored data set.

Moreover, the optimization function can be amended to take further advantageous features into account. In particular, the subset can be selected by further optimizing over a determination accuracy of the determination model with respect to the subset of molecule characterizing parameters. For example, for this case the optimization function can be defined as

$$O ( {X}_{i , f}^{scr} , {X}_{j , f}^{tr} ) = \frac{1}{S} \cdot {\sum }_{f , i , j} {c}_{f} \cdot D ( {X}_{i , f}^{scr} , {X}_{j , f}^{tr} ) + λ \cdot \frac{1}{T} {\sum }_{j} D * ( F ( {X}_{j , f}^{tr} ) , {Y}_{j}^{tr} )$$

Wherein

$$F ( {X}_{j , f}^{tr} )$$

refers to the technical application properties determined with the determination model that is trained with a respective training data set based on a respective selected subset,

$${Y}_{j}^{tr}$$

refers to the technical application properties for a respective molecule, T is the number of data points in the explored data set, D* is a generic distance measure between the technical application properties determined for the molecules of the explored data set with the determination model that is trained and the technical application properties of the molecules of the explored data set, and the parameter A refers to a weighting of the similarity between technical application properties determined with the determination model and the technical application properties of the explored data set. Moreover, the subset can be selected by further optimizing over an applicability of the determination model in the unexplored data set. In this case, the optimization function can be defined as

$$O ( {X}_{i , f}^{scr} , {X}_{j , f}^{tr} ) = \frac{1}{S} \cdot {\sum }_{f , i , j} {c}_{f} \cdot D ( {X}_{i , f}^{scr} , {X}_{j , f}^{tr} ) + λ \cdot \frac{1}{T} {\sum }_{j} D * ( F ( {X}_{j , f}^{tr} ) , {Y}_{j}^{tr} ) + μ \cdot \frac{1}{S} \cdot {\sum }_{i} G ( F ( {X}_{i , f}^{scr} ) , {Y}_{j}^{tr} )$$

Wherein

$$F ( {X}_{i , f}^{scr} )$$

refers to the technical application properties determined with the determination model that is trained,

$${Y}_{j}^{tr}$$

refers to the technical application properties for a respective molecule, G is a function that evaluates the applicability of the determination model in the unexplored data set, S is the number of data points in the unexplored data set, and the parameter u refers to a weighting of a measure of applicability of the determination model in the unexplored data set. However, also other mathematical formulations can be suitable as optimization functions or for deriving a respective subset of characterizing parameters for training the determination model.

The respectively selected subset of characterizing parameters then allows to define a training data set for parameterizing the determination model, wherein the training data set comprises the technical application properties of at least two of the explored molecules and the characterizing parameter values associated with the at least two explored molecules that refer to the selected subset of characterizing parameters. Known machine learning and training techniques can then be utilized to parameterize the determination model based on the training data set. The such trained determination model can then be provided and utilized, for example, by the target molecule determination method as described above with respect to FIG. 1.

In the following preferred examples of the above described method and the corresponding apparatus will be described in more details. In particular, the method, for example, as described above can be utilized to determine molecules for target technical application properties that belong at least to the group of a) mechanical properties, like adhesion, tensile-strength, stiffness, hardness, shrinkage, elongation, split-tear, tear-strength, rebound, compressibility, abrasion, spillage, morphology, haptic properties, stress at break, elongation at break, granulometry, degree of filling, b) optical properties, like coloration, turbidity, opaqueness, lucidity, reflection, appearance, absorbance, scattering, color strength, cloud point, matting degree, optical density, spectra, refractive index, c) physicochemical properties, like density, viscosity, K-value, molar weight, dispersity/molar mass distribution, particle size distribution, solubility, partition coefficients, interfacial properties, surface tension, dispersibility, storage stability, odor, segregation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, charge density, dart drop, melt volume rate, flowability, tear propagation resistance, sealing strength, permeation, d) chemical properties, like functional group count, atom type count, functional group density, atom type density, chemical resistance, reaction timing, demolding time, growing, hard/soft segment content, crystallinity, reaction temperature, reaction pressure, decomposition, thermal decomposition, photodegradation, acidity, pKa, pH, car-bon footprint, production costs, waste formation, moisture/water content, flammability, burning rate, self-ignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value, and e) biological properties, like biodegradability, biological resistance, toxicity, biotransformation, ecotoxicology, sensitization, bacterial count, enzyme activity, distribution in environment, bioaccumulation.

Thus, the method can be applied in a plurality of technical fields, like, agricultural molecules, coatings, dispersions, structural molecules, soluble molecules, polymer foams, e.g. for thermal and acoustic insulation, shoe and automotive applications.

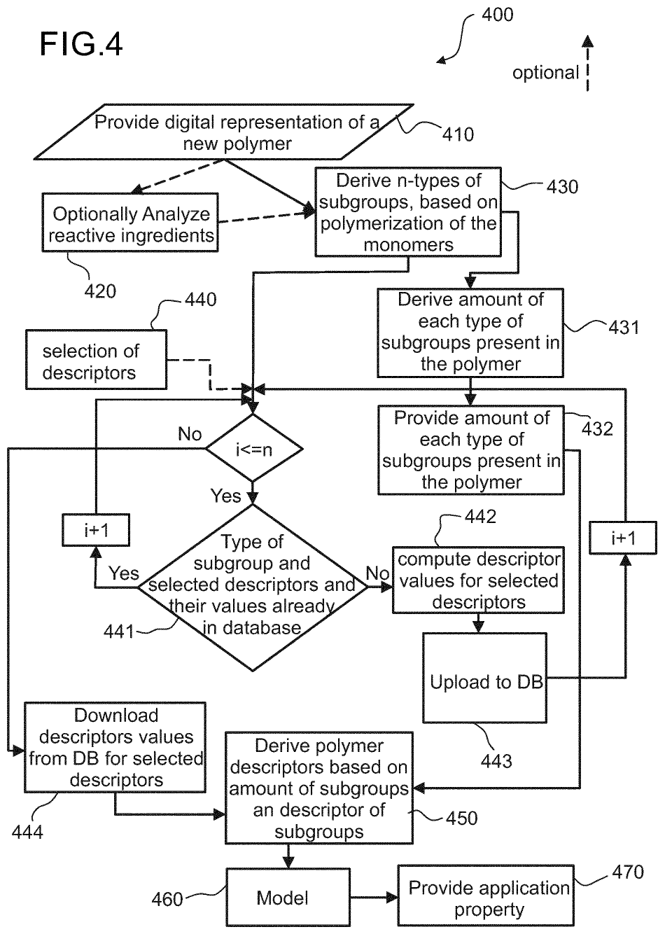

An exemplary embodiment of a method utilizing a determination model trained as described above for determining a technical application property can consist out of steps described in the following. In particular, such a method can be utilized for determining the technical application property in a method as described with respect to FIG. 1 in an embodiment in which the molecule is a polymer. A schematic and exemplary flowchart of the exemplary embodiment of the method 400 is show in FIG. 4. In a first step 410 a digital representation indicative of a polymer is provided. The digital representation can directly comprise polymer characterizing parameters, in particular, polymer descriptors, wherein in this case the steps shown in FIG. 4 until step 450 can be omitted. However, in many cases the polymer characterizing parameters first have to be determined based on the provided digital representation referring in such a case, for example, to a synthesis specification for the synthesis of the polymer. Generally, the digital representation can comprise or allow to derive any one or more of the following information an amount of monomer components; an amount of non-monomer components, like initiators, fillers, additives; a reaction condition, like temperature, vessel, pressure, stirring rate; condition profile, e.g. temperature profile, pH value, solvents; feed profiles; type of polymerization, e.g. radical, cationic, anionic, polycondensation, polyaddition, polyether formation; post-processing, like amount of components, conditions, as well as temperature and feed profiles; type of post-processing, e.g. radical, cationic, anionic, polycondensation, polyaddition, polyether formation; chemical information on components, like mixtures, connectivity of non-polymeric pure compounds, composition of polymeric pure compounds on the basis of subgroups, connectivity of the monomers associated to the subgroups in the polymeric pure components; for bock-co-polymers also information, in which block each monomer and reactive prepolymer is incorporated; for structures/layered materials and composites also information in which phase/layer each component is included. If such information is not provided by the digital representation directly, in optional step 420, the reactive components and subgroups can also be derived from the digital representation, for example, from the synthesis specification.

If the provided information indicates the presence of a mixture, then in a following step the mixture is decomposed into its pure components and each polymer component is treated as input polymer. Moreover, the polymer composition can also be transformed into mol %, if necessary.

In the next step 430 the polymerizable components can be transformed into subgroups, e.g. repeating units, and the subgroups are determined as different types. For example, polymerizable subgroups can be determined based on connectivity information of non-polymeric pure compounds by using SMARTS, for instance, via KNIME workflow. Also connectivity information of all possible subgroups can be derived from connectivity information of non-polymeric pure compounds by using reaction SMARTS, for example, also via KNIME workflow

After the subgroups and their types have been determined, in step 440 the type of characterizing parameters that should be utilized can be provided. However, the characterizing parameters can also be determined without first selecting the type of the subgroups. In order to decrease the computational resources for the method it is preferred that in a step 441 it is determined whether subgroup characterizing parameters associated with a respective type of subgroup are already stored in a database, for example, it can be determined if entries for subgroups with identical connectivity information already exist in the database. If this is the case the respective associated subgroup characterizing parameter can be directly downloaded, for example, in step 444. If the determined type of subgroup is not stored on the database the subgroup characterizing parameter that are associated with a respective type of subgroup can be determined, for example, in step 442. For example, either a 3D structure of respective types of subgroups can be derived based on connectivity information and an automatically computation of subgroup characterizing parameter can be started using, for instance, a computer cluster, or already existing machine-learning determinations can be utilized as subgroup characterizing parameter. Generally, it is preferred that if computations on new subgroups are necessary, in step 443, the results are stored in the database after the computations are finished. Optionally further subgroup characterizing parameters can be provided from a topological analysis of the subgroups, a quantum chemical computation, a molecular dynamics computation, coarse-grained methods, finite-element computations and kinetic simulations. In particular, polymer reaction engineering methods can be used to derive subgroup characterizing parameters that allow to take into account a microstructure of the polymer.

In step 431 the amount of subgroups, i.e. of each type of subgroup, is determined, for example based on the provided synthesis specification for the polymer. For example, the amount can be determined by counting an amount of polymerizable groups per polymerizable component, optionally, including prepolymers. In this case, information on polymerizable groups can be derived from non-polymeric components and the such determined amount can be added to a count of the number of, optionally, non-polymerized, polymerizable groups of the subgroups for polymeric components based on the composition of the polymeric components to determine a resulting amount. Further, it is preferred that the amount of polymerizable groups originating from agents used for post-processing after polymerization is removed from the resulting amount. In step 432 the such determined amount of subgroups can be provided and, for example, saved on the database. Before further processing the determined amount of subgroups, subgroups which are completely represented by other subgroups can be removed. Moreover, subgroups, which have the same connectivity, can be merged.

Optionally the derived amounts of subgroups can be used for a further interpretation of the polymer composition. For example, a total number of polymerized functional groups, e.g. double bonds, amine groups, alcohols groups, thiol groups, carboxylic acid groups, isocyanate groups, epoxide groups, and formed functional groups, e.g. amid groups, ester groups, thioester groups, urea groups, urethane groups, thiourethane groups, ether groups, can be determined. Also the molar weighted total number of polymerized functional groups, the mass weighted total number of polymerized functional groups, the total number of residual functional groups, e.g. double bonds, amine groups, alcohol groups, thiol, groups, carboxylic acid groups, isocyanate groups, epoxide groups, the molar weighted total number of residual functional groups, the mass weighted total number of residual functional groups, the sum of all residual functional groups, the ratio between functional groups after polymerization, the number of crosslinks in polymer, the molar fraction of crosslinks in polymer, optionally, with mass-weighting as well, the average number of atoms per subgroup, optionally, per weight as well, the average number of non-H-atoms per subgroup, optionally, per weight as well, the average number of bonds per subgroup, optionally, per weight as well, the average number of bonds between non-H-atoms per subgroup, optionally, per weight as well, the average number of rotors per subgroup, optionally, per weight as well, the average number of rotors between non-H-atoms per subgroup, optionally, per weight as well, the average number of rings per subgroup, optionally, per weight as well, the average polar surface areas per subgroup, optionally, per weight as well, the average refractivity per subgroup, optionally, per weight as well, the total number of blocks, the molar size of first block, the molar size of last block, the HLB value of polymer, optionally, with area weighted HLB value, the HLB value of block with lowest HLB value, optionally, with area weighted HLB value, the HLB value of block with largest HLB value, optionally, with area weighted HLB value, the HLB value of first block, optionally, with area weighted HLB value, the HLB value of last block, optionally, with area weighted HLB value, the mass of first block, the mass of last block, the area of block with lowest HLB value, the area of block with largest HLB value, the difference of the HLB values of the blocks, optionally, with area weighted HLB value, the hydrophilic area of the polymer, the lipophilic area of the polymer, the number of arms for ring-opening-polymerization, or the length of arms for ring-opening-polymerization can be determined.

In step 450 the determined amount and type of the subgroups and the associated subgroup characterizing parameters can be utilized to compute the polymer characterizing parameters. For example, the polymer characterizing parameters can be determined by one or more of molar weighted, e.g. arithmetic, harmonic or logarithmic, averaging, mass weighted, e.g. arithmetic, harmonic or logarithmic averaging, volume weighted, e.g. arithmetic, harmonic or logarithmic, averaging, surface area weighted, e.g. arithmetic, harmonic or logarithmic, averaging of the associated descriptors of the subgroups. Moreover, the polymer characterizing parameters can be determined by determining from the associated subgroup characterizing parameters one or more of a molar weighted standard deviation, a mass weighted standard deviation, a volume weighted standard deviation, a surface area weighted standard deviation, a molar weighted maximum value, a mass weighted maximum value, a volume weighted maximum value, a surface area weighted maximum value, a molar weighted minimum value, a mass weighted minimum value, a volume weighted minimum value, a surface area weighted minimum value, a molar weighted sum, a mass weighted sum, a volume weighted sum, a surface area weighted sum, and a maximal difference.

In step 460 the derived or provided polymer characterizing parameters can then be provided to the trained determination model for determining the application property. Generally, as already described above for determining of different polymer properties also differ-ently trained determination models can be utilized. The determination model is trained utilizing, for example, a training data set determined based on an unexplored data set and an explored data set, as described in detail above, with respect to FIGS. 2 and 3. The determination model can generally refer to sparse, e.g. Splines, LASSO regression, PLS, and non-sparse, e.g. ridge regression, tree methods, kernel based methods, statistical learning models for relating the polymer characterizing parameters to application properties of in-terest. Moreover, the determination model can further provide a reliability estimation of the determined technical application property depending on the respective used determination model. In step 470 the determined technical application property can then be provided to a user, for example, via a user interface, or can further be utilized to be compared with a target technical application property to determine a target polymer as target molecule as describe with respect to FIG. 1. In a further application an embodiment of the above described method can be utilized for virtual screening using, for example, one of composition optimization, Pareto optimization, low-dimensional visualization of screened recipes, fea-ture selection for maximal applicability domain of model, and applicability domain checker for new recipes on descriptor level.

Preferably the molecule characterizing parameters, in the above example polymer characterizing parameters, utilized in the above described method originate from quantum chemical computations with solvation treatment. Quantum chemical computations scale very un-favorable with the system size, which makes computations on polymers or shorter monomer sequences impractical. This obstacle is solved by the above method that comprises cutting the polymer at preferably non-polarized bonds into subgroups. The resulting subgroups have a similar size than the monomers and the characterizing parameters can be calculated using quantum chemical methods.

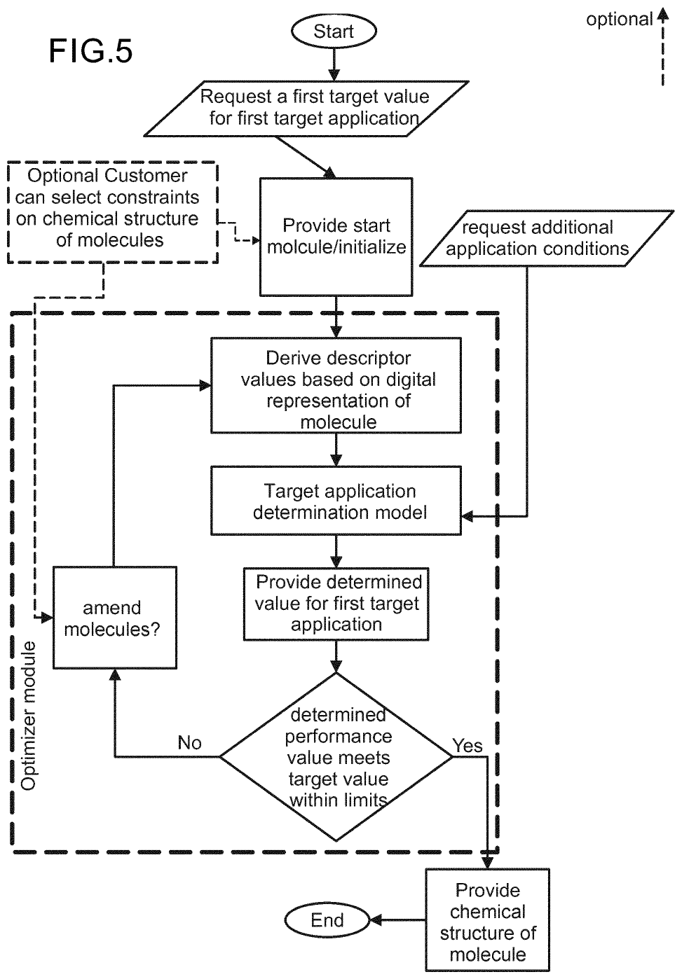

In the following, a more detailed preferred example of a preferred embodiment of a method utilizing a determination model trained as described above is described. A schematic and exemplary flow chart of an exemplary and preferred embodiment of the method is provided by FIG. 5. In this exemplary embodiment, the method starts with requesting, for instance, via a user interface, a target value for a target application. Moreover, in a next step, the optimization is initialized by providing a potential target molecule, for instance, by providing a synthesis specification and/or molecular structure, i.e. a start synthesis specification. Optionally, constraints on the chemical structure of the potential target molecule can be taken into account in this process, for instance, if a user provides such constraints. The constraints can refer, for instance, to constraints in the production of a molecule, in the starting substances that should be used for synthesizing the molecule, etc. Moreover, additional application conditions can be requested being in particular indicative of further possible information with respect to the target molecule that should be fulfilled. Based on the above steps, the optimization for determining the target molecule, i.e. the target synthesis specification and/or molecular structure, can be initialized. In a first step of the optimization, molecule characterizing parameter values can be derived from the provided start potential target molecule, for example, as described in detail with respect to FIG. 4 for a polymer. However, the deriving of the molecule characterizing parameters can also refer to accessing a storage on which respective molecule characterizing parameters for the respective potential target molecule are already stored. Moreover, if the provided digital representation of the potential target molecule already comprises the molecule characterizing parameters, this step can also be omitted. Based on the requested additional application conditions, a respective determination model can be provided. Based on the provided determination model and the digital representation of the potential target molecule, a value for the target technical application property for the potential target molecule can be provided. In a next step it is determined if the determined performance value, i.e. the determined technical application property, meets the target value within predetermined limits. If this is not the case, i.e. if this condition is not fulfilled, the formulation of the potential target molecule is amended and a new target molecule is determined optionally taking into account the constraints previously provided. The iteration can then start anew for the new potential target molecule. If at one point the determined performance value meets the target value within limits, i.e. if the respective condition is fulfilled, the potential target molecule is determined as the target molecule and provided, for example, to a user or to a control unit for producing the respective determined target molecule.

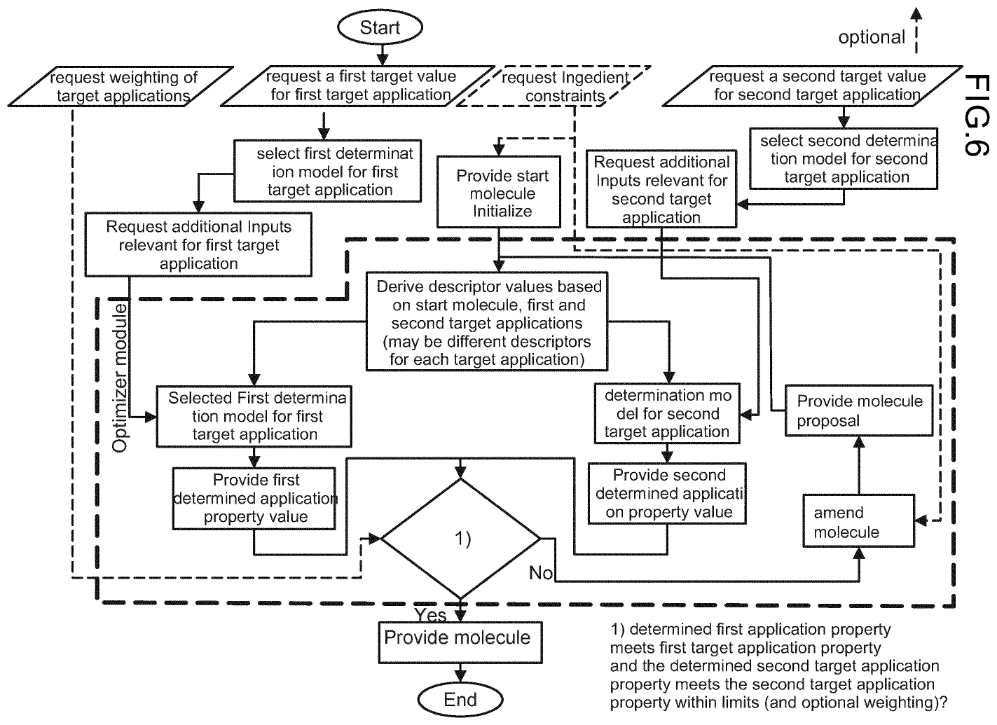

FIG. 6 shows schematically and exemplarily a further preferred embodiment of the above method for determining a target molecule with a predetermined first target technical application property, wherein in this embodiment in addition to the first target application property it is desired that the target molecule also fulfills a further second target value, i.e. target technical application property. The additional target technical application property can refer also to any technical application property. In particular, the method follows the same principles as described above with respect to FIG. 5. However, due to the additional target value, additional conditions have to be met during the optimization. Thus, in the following only the main differences with respect to the method as described above will be pointed out. In particular, in this preferred embodiment, the optimizer module does not only optimize over the first target value, i.e. over the first target application property, but also over the second target value. Preferably, also for the second target value a determination model adapted for determining a value for the technical application property based on molecule physicochemical parameters is utilized. Thus, in addition to the method as described above for the second target application a second determination model is provided that allows to determine an application property value based on the molecule physicochemical parameters for the second target application. The second determination model can, for instance, be based on the same algorithm as the first determination model, and is trained in accordance with the same principles as described above only with respect to the determination of another technical application property. The comparison then refers to not only determining whether the determined first application property meets the target application property within limits, but also whether the determined second application property value meets the target second application property value within limits. Predetermined rules can be utilized that determine for which cases the iteration is continued, i.e. a new potential target molecule is provided and for which conditions the potential target molecule is determined as the target molecule. For example, a user can predetermine weights for weighting to which ex-tents which of the conditions has to be met. For instance, it can be more important for a user that the first technical application property is met, whereas the other target application property is not so important. In this case, either the limits within which the second target application property can be met can be set broader or the meeting of this condition can be weighted less strongly. If at one point of the iteration it is then determined that the conditions are met and fulfil the predetermined rules the respective potential target molecule can be determined as target molecule and provided as output to a user or can be utilized to generate a control file for producing the respective target molecule.

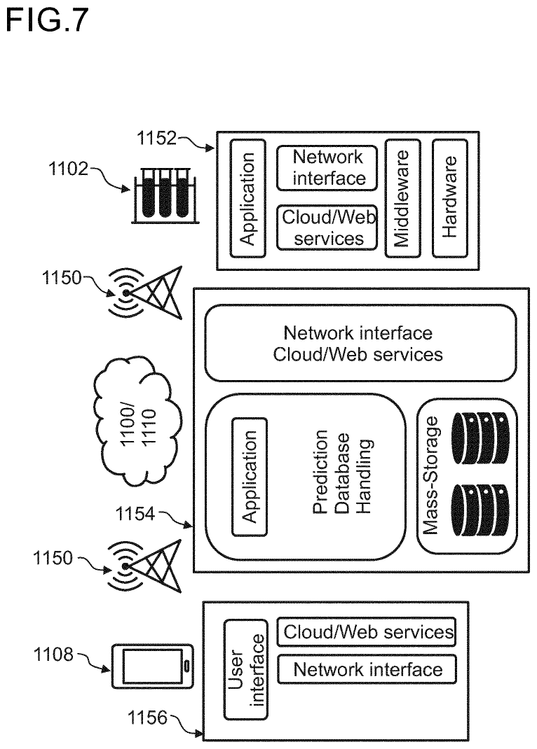

FIG. 7 illustrates a block diagram of an exemplarily system architecture of an automated laboratory system 1000 for synthesizing a molecule with a laboratory equipment control device 1102, a network 1150 and the synthesis specification, i.e. recipe, module 1100/1110, and a client device 1108. The automated laboratory system includes a laboratory equipment control device layer 1152 as part of the laboratory equipment control device 1102 as well as a synthesis specification module layer 1154 associated with the synthesis specification module and a remote control or client layer 1156 associated with the client device 1108. The laboratory equipment control device layer can be split into several hier-archical layers: the hardware, the middleware and the interface layer. The hardware layer relates to hardware resources such as sensors and actuators, in particular for controlling a synthesis of a molecule. The middleware relates to any of the known middleware for laboratory or plant synthesis operations. One example is LABS/QM, providing different abstrac-tions to hardware, network and operating system such as low-level device control and message passing. The communication layer relates to communication protocols, wherein the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The synthesis specification module layer 1154 may include: a mass storage layer, the computing layer, the interface layer. The storage layer is configured to provide mass storage for the data-driven determination model for providing a synthesis specification of a molecule based on a technical application property, as described in detail above. In particular, the functions performed by the apparatus, as described above, can be provided as program code means stored on the mass storage. Furthermore, synthesis specifications for a plurality of molecules can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on target properties. Such functionalities can include determining based on a target technical application property and the determination model a digital representation of a target molecule, generating a synthesis specification from the digital representation of the target molecule, and providing the synthesis specification as control data, i.e. control signal, to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the synthesis specification module layer 1154 or the laboratory equipment control device layer 1152. Users may be provided with a UI for selecting a target technical application property and a target value for this property, the target value may also comprise a range of the technical application property. In other examples, the users may be provided with a UI for selecting more than one technical application property and respective values. The applications may be configured for users to monitor and control the laboratory equipment control device and the operation remotely. In other examples, the client device layer and the synthesis specification module layer may be integrated into one device. The alternatives described here are only for illustration purposes and should not be considered limiting.

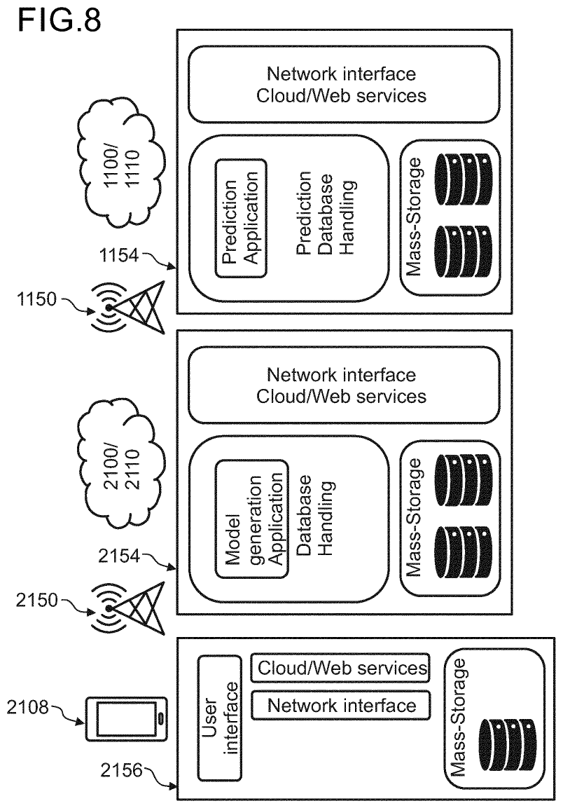

FIG. 8 illustrates a block diagram of an exemplarily system architecture of a system and apparatus for generating a determination model for determining a technical application property, a network 2150 and a model generating module 2100/2110 that can be regarded as or comprising a training model apparatus, a synthesis specification module 1100/1110, and a client device 2108. The system for generating a determination model includes a model generating module layer 2154 as part of model generating module and a client layer 2156 associated with the client devices 2108.

The model generating module layer 2154 may include: a mass storage layer, a computing layer, an interface layer. The storage layer is configured to provide mass storage for the data-driven determination model as described above. Furthermore, the mass storage is configured for storing synthesis specifications for molecules and technical application properties. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a determination model for determining properties of molecules. Such functionalities may include receiving for at least two previously explored molecules their respective digital representation associated with the molecule and measurement data of at least one technical application property for each of the at least two previously explored molecules, receiving at the model generating module the digital representation of at least one unmeasured molecule, training the model according to the above described training principles based on the digital representation of the at least two previously explored molecules, the at least one technical application property for each of the at least two previously measured molecules, and, preferably, a similarity measure between the digital representation associated with the at least two previously explored molecules and the respective digital representation associated with the at least one unmeasured molecule, and providing via an output interface the determination model for the technical application property. The model generating module layer may be configured for deploying the generated model and the synthesis specification database to the synthesis specification module layer. This may include storing the generated model and the synthesis specification database in the mass storage devices associated with the synthesis specification module.

The model generating module layer may further be configured for determining a digital representation of the molecule associated with the synthesis specification from the synthesis specification. The digital representation may include a set of molecule characterizing parameters and molecule characterizing parameter values associated with a synthesis specification of each explored molecule. One way of deriving these molecule characterizing parameters can be to apply the SMILES algorithm or any other already above described principle. In case, where the model is generated based on the digital representation derived from the synthesis specification, a relation between the synthesis specification and the characterizing parameters may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain synthesis specifications for molecules, and for at least two molecules at least one technical application property. The client layer further provides an interface for end-users. For end-users, the client layer 2156 may run client side Web applications, which provide interfaces to the model generation module layer 2154 or the mass storage devices associated with the client layer. Users may be provided with a UI for selecting a technical application property. The user may further be provided with a UI for selection of the molecule data and the technical application property data associated with the molecule data.

The user interface may also provide an option for uploading the selected data to the model generating module layer and optionally an option to initiate model generation.

Other variations to the disclosed embodiments can be understood and effected by those skilled in the art in practicing the claimed invention, from a study of the drawings, the dis-closure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the digital representation and the target technical application property, the determining of the technical application property, the comparing of the technical application property, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would under-stand that the structure of an executable component may include software objects, rou-tines, methods, and so forth, that may be executed on the computing system. This may include both an executable component in the heap of a computing system, or on computer-readable storage media. The structure of the executable component may exist on a com-puter-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other in-stances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communica-tions connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile tel-ephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configura-ble computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to a method for determining a synthesis specification. A target property and a digital representation of a potential synthesis specification is provided. Then a model is utilized for determining a property of a potential target molecule. The model has been parameterized based on an explored data set and an unexplored data set. The explored data set comprises a property for a plurality of explored molecules and molecule characterizing parameter values for the plurality of explored molecules. The unexplored data set comprising characterizing parameter values for a plurality of unexplored molecules. The determined property of the potential target molecule is compared with the target property. Based on the comparison, either i) the potential target molecule is determined as the target molecule, or ii) a new potential target synthesis specification is determined and the determination of the property repeated. The determined target molecule is then provided.

1. A computer implemented method for determining a synthesis specification and/or a molecular structure of a target molecule, in particular, target polymer, comprising a target technical application property, wherein the method comprises:
providing a target technical application property,
providing a digital representation of a potential target molecule indicative of or associated with characterizing parameters,
utilizing a trained determination model for determining a technical application property of the potential target molecule, wherein the trained determination model has been parameterized based on an explored data set and an unexplored data set such that the trained determination model is adapted to determine a technical application property of a molecule based on one or more molecule characterizing parameters of the molecule, wherein the explored data set comprises a) a technical application property for a plurality of explored molecules and b) a plurality of molecule characterizing parameter values for a plurality of molecule characterizing parameters associated with synthesis specifications and/or molecular structures corresponding to each of the plurality of explored molecules, and the unexplored data set comprising a plurality of molecule characterizing parameter values for a plurality of molecule characterizing parameters associated with synthesis specifications and/or molecular structures corresponding to a plurality of unexplored molecules, comparing the determined technical application property of the potential target molecule with the target technical application property and, based on the comparison, either i) determining the potential target molecule as the target molecule, or ii) providing a new potential target molecule and repeating the determination of the technical application property utilizing the new potential target molecule,
providing the determined target molecule.

2. The method according to claim 1, wherein the providing of the determined target molecule comprises generating control signals based on the target molecule and providing the control signals, wherein the control signals are configured to control a production system for producing the target molecule.

3. The method according to claim 1, wherein the parameterizing of the machine learning based determination model is based on determining a similarity measure between members of the explored data set and members of the unexplored data set.

4. The method according to claim 3, wherein the parameterizing of the machine learning base determination model comprises selecting a subset of the molecule characterizing parameters of the explored data set as training molecule characterizing parameters based on the similarity measure, wherein the parameterization of the machine learning based determination model utilizes a training data set comprising from the explored data set a) the technical application property for at least two explored molecules of the plurality of explored molecules, and b) molecule characterizing parameter values for the training molecule characterizing parameters associated with synthesis specifications and/or molecular structures corresponding to each of the at least two explored molecules.

5. The method according to claim 4, wherein the subset of the molecule characterizing parameters is selected based on an optimization of the similarity measure with respect to the subset of the molecule characterizing parameters between the members of the unexplored data set and the explored data set.

6. The method according to claim 3, wherein the similarity measure is indicative of distances between the explored data set and members of the unexplored data set with respect to the subset of the molecule characterizing parameters.

7. The method according to claim 5, wherein the subset is selected by further optimizing over a determination accuracy of the determination model with respect to the subset of molecule characterizing parameters.

8. The method according to claim 5, wherein the subset is selected by further optimizing over an applicability of the determination model in the unexplored data set.

9. A computer implemented method for generating a machine learning based determination model such that the trained determination model is adapted to determine a technical application property of a molecule based on one or more molecule characterizing parameters, wherein a molecule characterizing parameter is associated with a synthesis specification and/or molecular structure of a molecule, wherein the method comprises:
providing an explored data set comprising a) a technical application property for a plurality of explored molecules and b) a plurality of molecule characterizing parameter values for a plurality of molecule characterizing parameters associated with synthesis specifications and/or molecular structures corresponding to each of the plurality of explored molecules,
providing an unexplored data set comprising a plurality of molecule characterizing parameter values for a plurality of molecule characterizing parameters associated with synthesis specifications and/or molecular structures corresponding to a plurality of unexplored molecules,
generating the trained determination model by parameterizing the machine learning based determination model based on the explored data set and the unexplored data set such that the trained determination model is adapted to determine a technical application property of a molecule based on one or more molecule characterizing parameters of the molecule,
providing the generated trained determination model.

10. A computer implemented method for determining on one or more molecule characterizing parameters for training a machine learning based determination model such that the trained determination model is adapted to determine a technical application property of a molecule based on the one or more molecule characterizing parameters, wherein a molecule characterizing parameter is associated with a synthesis specification and/or molecular structure corresponding to the molecule, wherein the method comprises:
providing an explored data set comprising a) a technical application property for a plurality of explored molecules and b) a plurality of molecule characterizing parameter values for a plurality of molecule characterizing parameters associated with synthesis specifications corresponding to each of the plurality of explored molecules,
providing an unexplored data set comprising a plurality of molecule characterizing parameter values for a plurality of molecule characterizing parameters associated with synthesis specifications and/or molecular structures corresponding to the plurality of unexplored molecules,
selecting on or more molecule characterizing parameters from characterizing parameters of the explored data set based on the explored data set and the unexplored data set, and
providing the selected molecule characterizing parameters.

11. An interface method for providing a target synthesis specification indicative of a target molecule comprising a target technical application property, wherein the interface comprises:
receiving, via an input unit, a target technical application property,
interfacing, via an interface unit, with a processor performing the method according to claim 1 for providing the target technical application property, and receiving the determined target synthesis specification, and
providing, via an output unit, the determined target synthesis specification.

12. An apparatus configured to perform the method according to claim 1 for determining a target synthesis specification indicative of a target molecule comprising a target technical application property, wherein the apparatus comprises:
an input interface configured to
i) provide a target technical application property, and
ii) provide a digital representation of a potential target molecule indicative of or associated with characterizing parameters,
a processor configured to
i) utilize a trained determination model for determining a technical application property of the molecule, wherein the trained determination model has been parameterized based on an explored data set and an unexplored data set such that the trained determination model is adapted to determine a technical application property of a molecule based on one or more molecule characterizing parameters of the molecule, wherein the explored data set comprises a) a technical application property for a plurality of explored molecules and b) a plurality of molecule characterizing parameter values for a plurality of molecule characterizing parameters associated with synthesis specifications and/or molecular structures corresponding to each of the plurality of explored molecules, and the unexplored data set comprising a plurality of molecule characterizing parameter values for a plurality of molecule characterizing parameters associated with synthesis specification and/or molecular structures corresponding to a plurality of unexplored molecules, and
ii) compare the determined technical application property of the potential target molecule with the target technical application property and, based on the comparison, either i) determining the potential target molecule as the target molecule, or ii) providing a new potential target molecule and repeating the determination of the technical application property utilizing the new potential target molecule, and
output interface configured to provide the determined target molecule and the corresponding determined target technical application property.

13. A computer program product for determining a target synthesis specification indicative of a target molecule comprising a target technical application property, wherein the computer program product comprises program code means for causing a computing system to execute the method according to claim 1.

14. Control signals generated utilizing the method of claim 2.

15. (canceled)
